(19) United States
(12) Patent Application Publication
(10) Pub. No.: US 2023/0054976 A1
(43) Pub. Date: Feb. 23, 2023

(54) METHODS FOR INHIBITION OF ALPHA-SYNUCLEIN mRNA USING SMALL MOLECULES

(71) Applicants: The Scripps Research Institute, La Jolla, CA (US); Rutgers, The State University of New Jersey, New Brunswick, NJ (US)

(72) Inventors: Matthew David DISNEY, Jupiter, FL (US); M. Maral MOURADIAN, Princeton, NJ (US)

(21) Appl. No.: 17/757,609

(22) PCT Filed: Dec. 18, 2020
(86) PCT No.: PCT/US2020/065901

§ 371 Date: Jun. 17, 2022

Related U.S. Application Data
(60) Provisional application No. 62/950,267, filed on Dec. 19, 2019.

(51) Int. Cl. A61K 31/4184

(52) U.S. Cl. CPC A61K 31/4184

(57) ABSTRACT

A sequence-based design has provided a group of small molecules that target the IRE structure and inhibit SNCA translation in cells, which are named synucleozid compounds. The synucleozid compounds have a diphenyloxo- or diphenylamino-benzimidazole or indole core with various terminal amino or heterocycle groups as substituents.

### CROSS-REFERENCE TO RELATED APPLICATION

This application claims the priority of U.S. provisional application Ser. No. 62/950,267, filed 19 Dec. 2019, the disclosure of which is incorporated herein by reference in its entirety.

### BACKGROUND

Human diseases are often caused by malfunctioning proteins with diverse functions, and yet only a small set of them can be drugged or targeted with a small molecule¹⁻². Indeed, a genome-wide analysis showed that only 15% of proteins are considered to be in druggable protein families, while the remaining 85% are considered “undruggable”¹⁻². Many of these undruggable proteins do not fold into defined structures or assume structures lacking pockets suitable for binding small molecules.³⁻⁴

One intriguing strategy to expand protein druggability, particularly for proteins with aberrantly high levels linked to disease, is to target their coding mRNAs and inhibit translation. Such an approach could be accomplished by defining structured regions in an mRNA, i.e., potential small molecule binding pockets, and then identifying lead small molecules that bind these structures; that is, a sequence-based design strategy.

α-Synuclein is a key protein in the pathogenesis of Parkinson's disease (PD) and other α-synucleinopathies based on genetic, neuropathology, cell biology and animal model studies⁷. This protein can oligomerize, misfold, and form fibrils that propagate across neurons in the brain and accumulate in Lewy bodies and Lewy neurites⁸⁻⁹. The expression level of α-synuclein is an important determinant of the rate of its fibrillization and neurotoxicity¹⁰⁻¹¹, as individuals with multiplication of the SNCA gene locus develop dominantly inherited PD and dementia with a gene dosage effect¹². Additionally, polymorphisms in the promoter region and in a distal enhancer element of the SNCA gene impact α-synuclein protein levels and elevate the risk of developing PD¹³⁻¹⁵.

Like about 85% of proteins, α-Synuclein is an intrinsically disordered protein (IDP) and is therefore difficult to target, owing to its lack of defined small molecule binding pockets. At the RNA level, however, SNCA mRNA displays a functionally important and structured 5′ untranslated region (UTR) with an iron responsive element (IRE) that regulates its translation (FIGS. 1A and 1B)¹⁸⁻¹⁹. The IRE is bound by iron regulatory protein (IRP) at low concentrations of iron. At high concentrations, IRP is bound by iron, freeing the mRNA to undergo translation²⁰⁻²¹. The presence of iron in Lewy bodies and translational control of α-synuclein via iron and the IRE support their collective roles in PD²²⁻²³. Thus, reducing the expression level of this protein is expected to be a disease modifying strategy¹⁶⁻¹⁷ (FIG. 1A).

Small molecules that target this RNA structure could be of value as probes that inhibit translation of α-synuclein, enabling the study of associated pathogenetic mechanisms. Further, such studies could provide new strategies for drugging “undruggable” proteins by targeting them at the RNA level.

An object of the present invention, therefore, is to develop methods for moderating α-synuclein expression through use of small molecules that are capable of binding with mRNA coding for α-synuclein. A further objective is to develop methods for such moderating involving use of small molecules that interdict the translation of an α-synuclein gene, SNCA to the protein. Another objective is to develop such methods involving small molecules that do not interdict other kinds of mRNA. Another objective is to manage the biosynthesis of α-synuclein. A further objective is to enable reduction of excessive α-synuclein biosynthesis. Yet another objective is to treat diseases associated with α-synucleic.

### SUMMARY

The present invention is directed to these and other objects through development of embodiments that affect α-synuclein biosynthesis and/or its expression and/or its concentration produced by cells having the SNCA gene. According to the invention, these other objects relate to embodiments of the invention directed to methods employing small molecules that have binding/complexing capability with mRNA transcribed from the SNCA gene. Additionally, embodiments of the invention are directed to selective engagement of the mRNA transcribed from the SNCA gene.

The method embodiments of the invention are directed to complexing, binding, inhibiting, reducing and/or modulating the production and/or cellular concentration of α-synuclein by binding SNCA mRNA with small molecules capable of affecting the expression of α-synuclein. According to the invention, the management of this expression can be accomplished by affecting the relationships among IRP, iron and the IRE of the SNCA mRNA.

According to invention, the embodiments of the methods of the invention are directed to use of a composition comprising a synucleozid compound comprising Formula I and pharmaceutically acceptable salts thereof:

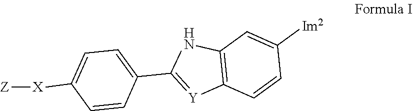

Embodiments of Formula I comprise those having substituents Z, X, Y and Im² as features of Formula I that promote the management of the expression of SNCA mRNA by Formula I. In particular, substituent X may be oxygen or NR² wherein R¹ may be hydrogen or methyl. Substituent Y may be nitrogen or CR¹ wherein R² may be hydrogen or methyl. Substituent Z may be hydrogen, methyl or the aromatic group:

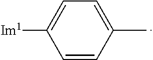

Each substituent Im¹ and Im² may independently be: imidazolyl, dihydroimidazolyl, imidazolinyl, pyrrolyl, pyrrolidinyl, cyano or guanidyl wherein guanidyl is a moiety of the structure:

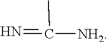

A preferred embodiment of Formula I comprises a syncleozid compound of Formula II and pharmaceutically acceptable salts thereof:

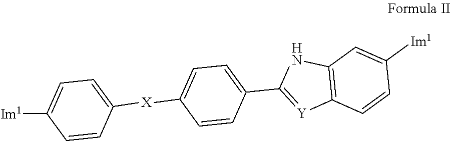

The substituents, X, Y, Im¹ and Im², are the same as given for Formula I.

Preferably. Im¹ and Im² are the same.

The present invention is further directed to embodiments of Formulas I and II formulated as a pharmaceutical composition that may be employed according to the methods of the invention. The pharmaceutical composition includes a pharmaceutically acceptable carrier.

The present invention is also directed to a method for treating a synucleinopathy disease comprising administering a synucleozid compound of Formula I or II or a pharmaceutical composition thereof.

The present invention is also directed to a method for complexing and/or binding SNCA mRNA comprising combining the mRNA with a synucleozid compound of Formula I or II or a pharmaceutical composition thereof.

The present invention is further directed to a method for reducing, inhibiting and/or modifying the production of α-synuclein protein by a cell carrying the SNCA gene by administering, dosing, infusing and/or applying to the cell a synucleozid compound of Formula I or II or a pharmaceutical composition thereof.

The present invention is further directed to a method for reducing, inhibiting and/or modifying translation of SNCA messenger RNA by combining in the presence of ribosomes, the messenger RNA with a synucleozid compound of Formula I or II or a pharmaceutical composition thereof.

According to the foregoing methods of the present invention, the cell carrying the SNCA gene and the corresponding messenger RNA may be a cellular culture or an in vitro RNA medium respectively or may be present in living tissue or in tissue of a mammalian animal or in tissue of a human.

The present invention is further directed to a composition of the synucleozid compound of Formula II and the pharmaceutically acceptable salt thereof.

### BRIEF DESCRIPTION OF THE DRAWINGS

FIG. 1A depicts IRE binding for Infoma hits and synucleozid compounds.

FIG. 1B depicts Inforna hits.

FIG. 1C depicts graph of relative expression of a synuclein influenced by synucleozid

FIG. 1D depicts a bar graph of LDR release.

FIG. 2A depicts expression of mRNA's detected by luciferase when contacted with certain small molecules.

FIG. 2B depicts graph of mRNA's through expression of β actin when contacted with certain small molecules.

FIG. 3A depicts secondary structure of 2-AP labeled RNA.

FIG. 3B provides a plot of 2 AP fluorescence change.

FIG. 3C is a plot of the affinity of synucleozid for SNCA IRE mutants.

FIG. 4A shows designed ASOs.

FIG. 4B shows relative cleavage of SNCA IRE

FIG. 4C shows scheme and normalized fold change.

FIG. 5A shows an ASO Bind Map.

FIG. 5B shows expression of SNCA in cells.

FIG. 6A shows how synucleozid could affect loading of SNCA mRNA

FIG. 6B shows a representative absorption trace.

FIG. 6C shows percentage of SNCA mRNA present in certain fractions.

FIG. 7A illustrates how synucleozid could affect abundance of IRPs.

FIG. 7B shows that SNCA mRNA was pulled down from treated cells. depicts hypothesis and experimental results for synucleozid IRP interaction.

FIG. 8A depicts up and down regulated protein expression under scrambled control.

FIG. 8B depicts up and down regulated protein expression un synucleozid or vehicle control.

Fig S2 depicts protein modulation with synucleozid A treatment.

Fig S3 depicts synucleozid A effect on SNCA transcription (no effect).

Fig S4 depicts effect of synucleozid A on translation of SNCA mRNA.

Fig S5A depicts cell viability dosed with synucleozid A (MTS assay).

Fig S5B depicts cell viability dosed with synucleozid A (LDB release.

Fig S6 depicts structures of IRE's in mRNAs of several human genes including SNCA.

Fig S7A depicts competitive binding assay for synucleozid A and several mRNAs.

Fig S7B depicts assay for synucleozid A and additional mRNA's.

Fig S8 depicts secondary structures of the RNA competitors used in assay of Fig S7.

Fig S9 depicts the thermal melting experiments with synucleozid A.

Fig S10A shows activity of synucleozid derivatives.

Fig S10B illustrates activity of synucleozid derivatives in 2-AP assay.

Fig S10C illustrates activity of synucleozid derivatives for inhibition of α-synuclein expression.

Fig S11A shows designed ASO hybridized to A bulge and binding of synucleozid to predicted site.

Fig S11B shows binding to sites other than A bulge.

Fig S12 depicts the FRET based ASO bind map with synucleozid A.

Fig S13 depicts that synucleozid A has no effect on expression of IRP-1 or IRP-2.

Fig S14 depicts the gel mobility shift assays and graphs showing results of IRP displacement by synucleozid A.

Fig S15 depicts Western blot of α-synuclein in proteomics samples with synucleozid A and siRNAs.

Fig S16A depicts differential gene expression under the influence of synucleozid A and siRNAs (Scrambled results).

Fig S16B depicts differential gene expression under influence of synucleozid A versus vehicle.

### DETAILED DESCRIPTION

A sequence-based design known as the lead identification strategy, Infoma^{5,24} was employed to target the SNCA IRE. Infoma is built around a database of experimentally determined, privileged RNA fold-small molecule interactions. The interactions are both high affinity and selective. Inforna then searches an RNA target for druggability; that is, whether it houses an RNA fold in the database. The small molecules that bind this targetable fold are lead chemical probes that can be assessed for biological effects^{6,25-26}.

According to the present invention, Infoma provided a cohort of small molecules that bind folds present in the SNCA IRE. The most effective embodiments of compound. Synucleozid of Formulas I and II, selectively repressed α-synuclein translation in a neuronal cell line, as determined by proteome-wide studies, providing a cytoprotective effect. Cellular mechanistic studies revealed that embodiments of Synucleozid: (i) binds and stabilizes the SNCA IRE in cells at a specific structural element, as designed by Infoma; and, (ii) represses translation by stabilizing IRE, thereby causing an accumulation of ribosome precursors on the mRNA thereby reducing the amount of SNCA mRNA loaded into polysomes. The former mechanistic studies were enabled by a further embodiment of the present invention involving a mapping technique for studying molecular recognition of RNAs by small molecules both in vitro and in cells. This technique is known as an antisense oligonucleotide ligand binding site mapping (ASO-Bind-Map)²⁷.

### Definitions

Unless defined otherwise, all technical and scientific terms used herein have the same meaning as commonly understood by a person of ordinary skill in the art.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

It is also to be understood that as used herein and in the appended claims, the singular forms “a,” “an,” and “the” include plural reference unless the context clearly dictates otherwise, for example, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

At various places in the present specification substituents of compounds of the invention are disclosed in groups or in ranges. It is specifically intended that the invention include each and every individual subcombination of the members of such groups and ranges. For example, the term “C1-C6 alkyl” is specifically intended to individually disclose methyl, ethyl, propyl, isopropyl, n-butyl, sec-butyl, isobutyl, etc. For a number qualified by the term “about”, a variance of 2%, 5%, 10% or even 20% is within the ambit of the qualified number.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986). Int J. Pharm., 33, 201-217, incorporated by reference herein.

Each of the terms “halogen,” “halide,” and “halo” refers to —F, —Cl, —Br, or —I.

The term “nitrile” or “cyano” can be used interchangeably and refer to a —CN group which is bound to a carbon atom of a heteroaryl ring, aryl ring and a heterocycloalkyl ring.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound of the invention can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

The terms “prevent,” “preventing,” and “prevention” refer to the prevention of the onset, recurrence, or spread of the disease in a patient resulting from the administration of a prophylactic or therapeutic agent.

A “patient” or “subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

### Screening and Design of Synucleozid Compounds Targeting SNCA IRE

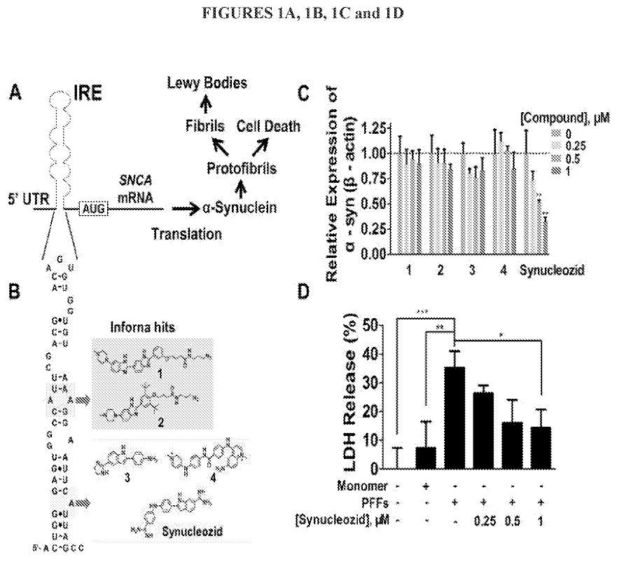

The translation of some α-synuclein isoforms is regulated by a three-dimensionally folded hairpin structure that is similar to iron responsive elements (IRE) in the 5′ UTR in its encoding mRNA (located in exon 1 upstream of the AUG start codon; FIGS. 1A and 1B)¹⁸⁻²¹. To determine the percentage of SNCA mRNA transcripts harboring the IRE, an RNA-seq analysis of SH-SY5Y cells which is a human dopaminergic neuroblastoma cell line commonly used to study the expression of α-synuclein was performed. Of the 13 SNCA mRNA transcripts²⁸, five transcripts passed the detection criteria for further analysis (at least 5 estimated counts in at least 47% of the samples²⁹; Table S1). Amongst these five transcripts, two (Transcript ID: SNCA-204 and SNCA-205) contain the targeted IRE sequence. SNCA-204 and SNCA-205 make up ˜50% of all SNCA mRNA species in SH-SY5Y cells (Table S1). Therefore, this IRE-like hairpin was found to be an attractive therapeutic target to reduce α-synuclein protein levels.

To explore such a strategy, design of small molecules was sought that bind the SNCA 5′ UTR IRE structure. Two different models of the SNCA 5′ UTR IRE structure have been reported in the literature, deduced from free energy minimization and/or comparative sequence analysis^{18,21,30}. The combined approach of evolutionary conservation and free energy minimization was used to refine the structure. The resulting model (FIG. 1B) is further supported by its structural conservation (91.6%) and the observation of multiple structure-preserving mutations in homologous SNCA sequences-spanning eutherian mammals, as explored using Rfam³¹ (Fig. S1). The structure is similar to that reported by Rogers et al.³⁰, except that the helix adjacent to the hairpin is slipped, affording remarkable similarity between the human SNCA and ferritin IRE structures. Interestingly, a potential compensatory mutation was identified in the conservation studies, supporting the revised model helix. In vitro mapping studies support that the two models may be in equilibrium. The lead identification strategy, Inforna^{5,24}, then identified five compounds that are privileged for two structural elements found in both models of the IRE, namely a 1×1 nucleotide internal loop and a 1-nucleotide bulge with GC and GU closing base pairs.

To investigate the likelihood that these structure form within IRE-containing species, the sequence conservation of their closing base pairs as well as sequence variations were evaluated. The 1-nucleotide bulge's closing pairs are 100% conserved in a MAFFT alignment of 55 sequences of eutherian mammals while one closing base pair of the internal loops is conserved 100% and the other <90%. To further investigate the sequence variation within the SNCA IRE, the NCBI database was queried and it was found that the highest population minor allele frequency (MAF) observed in any population, including 1000 Genomes Phase 3, ESP, and gnomAD, is equal to or less than 0.01 for all nucleotides, indicating high sequence conservation of SNCA IRE³².

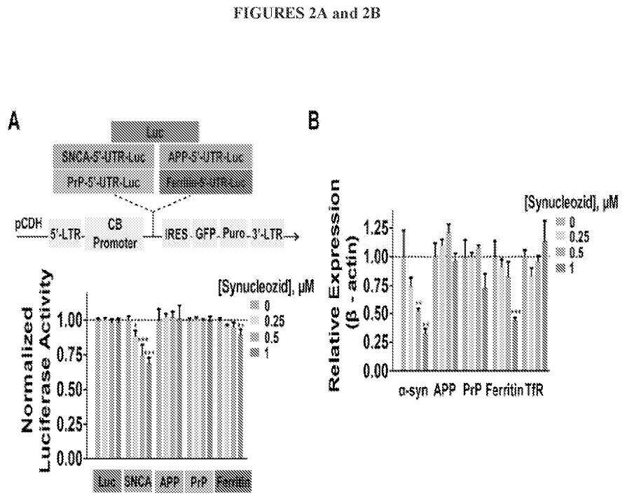

Five compounds were studied for inhibiting α-synuclein translation in SH-SY5Y cells (FIG. 1C). Of these, lead compound Synucleozid with guanidyl substituents as Im¹ and Im², (hereinafter synucleozid A) was found to bind the A bulge near the base of the IRE hairpin and reduced levels of α-synuclein in a dose-dependent manner with an IC₅₀ of ˜500 nM (FIGS. 1B, 1C and S2 compound labeled Synucleozid and hereinafter synucleozid A). In addition, precursor synucleozid 3 (FIG. 1B) displayed IRE hairpin binding results but were not as significant as those displayed by synucleozid A (FIG. 1B). To ensure that reduction of protein levels was due to inhibition of translation and not transcription, SNCA mRNA levels were measured by RT-qPCR upon synucleozid A compound treatment. Synucleozid A had no effect on the steady-state levels of SNCA mRNA (Fig. S3). In agreement with these cellular studies on endogenous α-synuclein protein and mRNA levels, Synucleozid A inhibited α-synuclein translation using a luciferase construct fused to the SNCA 5′ UTR, both in transfected SH-SY5Y cells (FIG. 2A) and in vitro (Fig. S4). Dose-dependent effects were observed in both systems, with 1 μM Synucleozid A inhibiting ˜40% of translation in cells. No inhibitory effect (cellular or in vitro) was observed for a construct in which the SNCA 5′ UTR was absent (FIGS. 2A and S4). Importantly, no toxicity was observed upon Synucleozid A treatment of SH-SY5Y cells, as determined by cell viability (MTS) and lactate dehydrogenase (LDH) release (Fig. S5).

To assess the biological effect of Synucleozid A on an α-synuclein-mediated phenotype, its ability was studied to confer cytoprotection against the toxicity of α-synuclein pre-formed fibrils (PFFs)³³. The PFFs are comprised of recombinant human α-synuclein that, when delivered to cells, seed the aggregation and fibrillization of soluble endogenous α-synuclein, triggering downstream cellular damage and toxicity that can be measured by LDH release. As expected for a compound that reduces levels of α-synuclein, Synucleozid A mitigated the toxicity induced by PFFs in a concentration dependent manner (FIG. 1D). Because of these favorable cellular properties of Synucleozid A, an indepth analysis of this compound and derivatives thereof was made to establish their mechanism of action and confirmed their direct engagement of the SNCA IRE.

### Compounds Complexing with SNCA mRNA

Embodiments of the method of the invention incorporate embodiments of synucleozid compounds having features of lead compounds synucleozid A and compound 3. FIG. 1B. These compound embodiments have been found to exhibit significant, selective binding with SNCA mRNA so that the translation of the mRNA and resulting expression of α-synuclein protein are suppressed, inhibited and/or moderated. Because expression and especially overexpression of α-synuclein protein is regarded as a causative factor for synucleinopathy disease such as Parkinson's disease, Dementia with Lewy Bodies and Multiple System Atrophy, reduction, moderation, inhibition and/or management of this expression according to the invention will abate meliorate or otherwise reduce the incidence, severity and/or consequences of these diseases.

The synucleozid compound embodiments useful for the methods of the invention comprise Formula I:

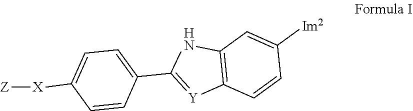

For Formula I, the substituent X may be O or N and the combination substituent Z-X may be hydroxyl, NH₂, NHMe, NMe₂ or any of the following X-phenyl moieties substituted by Im¹. The labels under each moiety indicate the identity of the substituent Im¹:

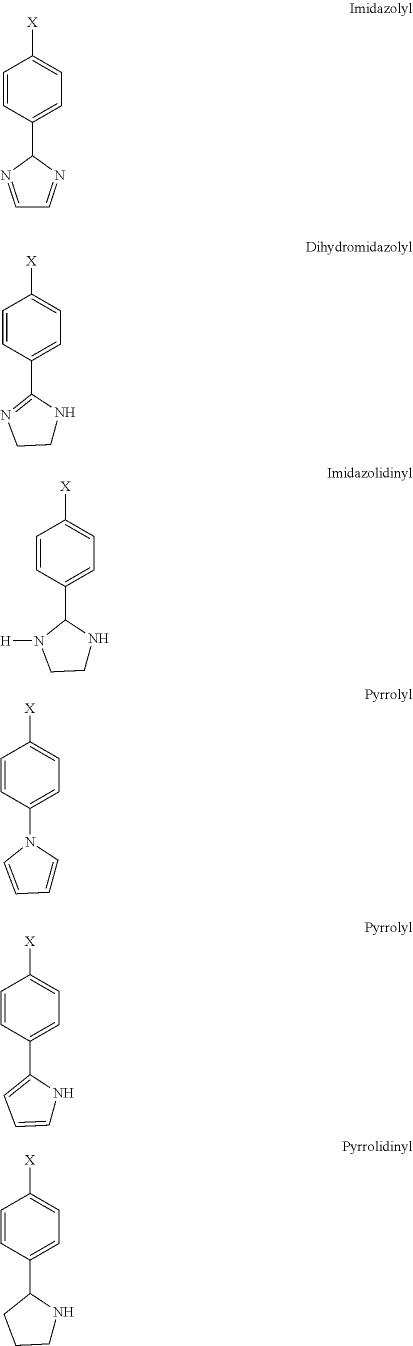

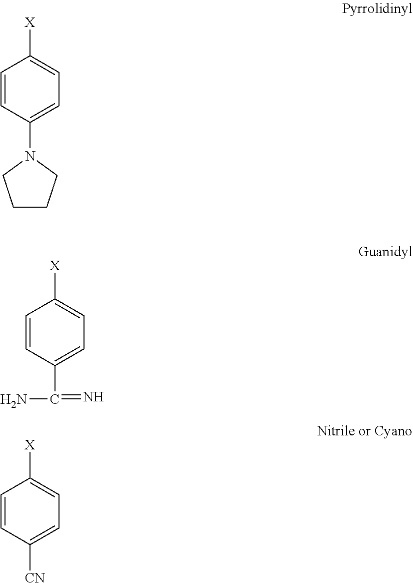

The Im² substituent has the same designations as the Im¹ substituent. Preferably, but not obligatorily. Im¹ and Im² may be the same.

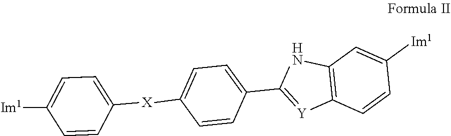

Preferred synucleozid compounds according to the invention have Formula II above. Formula II is a version of Formula I in which Z-X of Formula I is any of the X-phenyl moieties substituted by Im¹ as depicted above. More preferred synucleozid compounds of Formula II include those having Y as N or CH, X as oxygen or NH and the phenyl-Im¹ group (Z of Formula I) as phenyl substituted by imidazolyl, phenyl substituted by dihydroimidazolyl, phenyl substituted by guanidyl and phenyl substituted by cyano. More especially preferred synucleozid compounds include those of Formula II in which Im¹ and Im² are both dihydroimidazolyl, guanidyl or cyano, X is oxygen or NH and Y is CH or N. Most preferred synucleozid compounds include those of Formula II in which Im¹ and Im² are both dihydroimidazolyl. X is oxygen or NH and Y is CH or N.

Preferred exemplary synucleozid compounds are those of Formula II in which Im¹ and Im² are both imidazolyl, X is NH or O and Y is CH or N.

A most preferred exemplary synucleozid compound is Formula II in which Im¹ and Im² are both guanidyl, X is NH and Y is CH. This exemplary embodiment is synucleozid A.

### Biology of Synucleozid A

The following discussion of embodiments of the synucleozid compounds refers to Synucleozid with a capital S. This designation means and is the same as synucleozid A as described above as the most preferred exemplary compound of the synucleozid compound embodiments of Formula II.

The SNCA is not the sole mRNA expressed in the nervous system that contains an IRE in its 5′ UTR. Therefore, Synucleozid was studied to determine whether it also inhibits translation of these mRNAs, including amyloid precursor protein (APP), prion protein (PrP), and ferritin. Analogous luciferase reporter gene constructs were created for the APP. PrP, and ferritin 5′ UTRs (FIG. 2A top, and Supplementary Methods) and the effect of Synucleozid was studied on their translation in SH-SY5Y cells. In contrast to the luciferase reporter fused to the SNCA 5′ UTR, Synucleozid had no effect on translation of luciferase fused to the APP or PrP 5′ UTRs. A very small (˜10% inhibition), but statistically significant effect, was observed for ferritin upon treatment with 1 μM Synucleozid (similar to the percent inhibition observed for the SNCA 5′ UTR at a 4-fold lower concentration, 250 nM) (FIG. 2A bottom). It is not surprising that Synucleozid is selective for the SNCA 5′ UTR as the four UTRs studied have different secondary structures (Fig. S6).

Considering these positive results using luciferase constructs, the endogenous levels of APP, PrP, and ferritin were measured in SH-SY5Y cells upon Synucleozid treatment. In addition, the effect of the small molecule on the transferritin receptor (TfR) was also studied as it contains an IRE in its 3′ UTR³⁴ and because of its role in regulating the translation of mRNAs with IREs in their 5′ UTRs²¹. Mirroring the results observed using luciferase constructs, Synucleozid had no effect on protein levels of APP, PrP or TfR (FIGS. 2B and S2). Although no dose response was observed for its effect on ferritin, its levels were reduced by ˜50% at the highest concentration tested (1 μM; FIGS. 2B and S2). This reduction in ferritin levels could be due to an off-target effect and/or rescue of autophagic and lysosomal dysfunction observed in PD. This dysfunction has been linked to α-synuclein accumulation and α-synuclein-mediated disruption of hydrolase trafficking³⁵. As long-lived proteins, including ferritin, are degraded in the lysosome, rescue of lysosomal function by Synucleozid may account, in part, for reduction of ferritin levels. In support of this notion, a study in α-synuclein^{-/-} mice showed that α-synuclein impaired ferritinophagy and conversely that elimination of α-synuclein reduced ferritin levels³⁶.

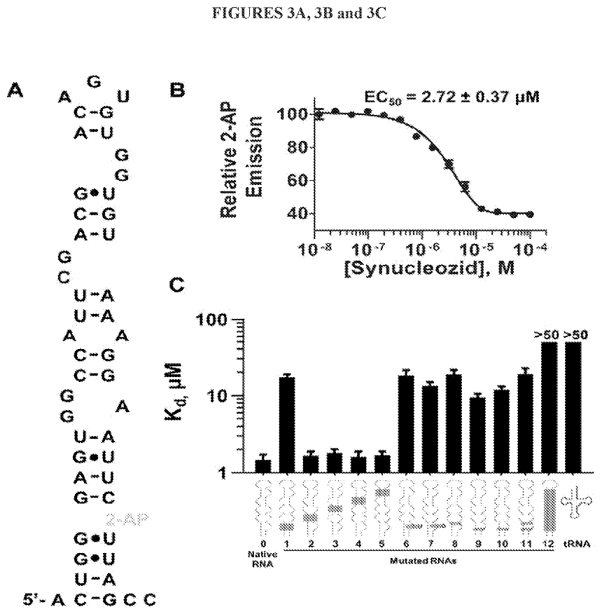

Next, the affinity of Synucleozid for its putative binding site in the IRE, the A bulge, was measured by replacing the bulge with the fluorescent adenine mimic 2-Aminopurine (2-AP) (FIG. 3A). The emission of 2-AP changes based on its microenvironment, particularly if it is stacked on neighboring bases³⁷, which can be altered upon ligand binding³⁸⁻³⁹. Indeed, Synucleozid decreased 2-AP emission with an EC₅₀ of 2.7±0.4 μM (FIG. 3B). Importantly, recovery of 2-AP emission was observed as a function of unlabeled SNCA IRE RNA (RNA-0) concentration, affording a competitive K_{d} of 1.5±0.3 μM (FIGS. 3C and S7A). That is, both 2-AP-labeled and native IRE RNA bind to Synucleozid with similar affinities. Therefore, 2-AP-labeled IRE RNA can serve as a useful model to assess Synucleozid binding and avidity.

To converge on the A bulge as Synucleozid's binding site and characterize it, competitive binding assays were completed with a series of unlabeled RNAs in which mutations were introduced (FIGS. 3C and S8) and 2-AP-labeled IRE RNA. For these investigations, each non-canonically paired region was systematically replaced with base pairs, generating RNA-1 to RNA-5 (FIGS. 3C and S8). Additionally, a mutational analysis for A bulge and its surrounding base pairs (RNA-6 to RNA-11; FIGS. 3C and S8) was completed but mutation of the A bulge to a C was not accomplished as it caused structural rearrangement of the neighboring paired region. If Synucleozid selectively binds to the 5′G_G/3′CAU (the A bulge and its closing pair), their mutation should all negatively impact binding. In contrast, mutation of the remaining non-canonically paired regions that are not putative Synucleozid binding sites should not significantly affect binding affinity.

The mutation of the A bulge to a base pair (RNA-1) or to a U or G bulge (RNA-6, 7) reduced Synucleozid avidity by 10-fold as compared to the native IRE (K_{d}˜20 μM; FIGS. 3C, S7B and S7C). Mutation of the closing base pairs (RNA-8 to 11) also reduced binding affinity, emphasizing the importance of the GC and GU closing base pairs in Synucleozid's molecular recognition of the native IRE (FIGS. 3C and S7C). The ferritin IRE has three U bulges and one C bulge (Fig. S6). This 10-fold weaker binding observed to the U bulge, which might in part be traced to Synucleozid's doubly charged nature at physiological pH, could contribute to the observed reduction of ferritin protein levels (FIG. 2). Notably, as discussed above, the decreased ferritin levels observed upon Synucleozid treatment could also be due to rescue of in α-synuclein-mediated lysosomal dysfunction, as reduced ferritin levels were observed in α-synuclein mice³⁶.

Replacement of all other non-canonically paired regions with base pairs (RNA-2 to 5) had no effect on Synucleozid binding, further indicating the selectivity of the compound for the A bulge. Two other RNAs were also studied in this competition assay. RNA-12 in which all internal loops and bulges, including the Synucleozid binding site, were replaced with base pairs, and tRNA. No significant recovery of the change in 2-AP emission was observed until >100 μM of either RNA was added (FIGS. 3C and S7A). To support these binding studies, thermal melting experiments were performed on the wild type A bulge IRE RNA and the corresponding fully paired RNA in the presence and absence of Synucleozid. The compound only stabilized the wild type IRE upon binding, decreasing its ΔG_{37°} from −2.91 to −3.23 kcal/mol and increasing its Tₘ by 3° C. (from 51.4 to 54.8° C.; Fig. S9). Taken together with the binding studies on IRE mutants, these mutational studies indicate that Synucleozid selectively recognizes the A bulge in the SNCA IRE, resulting in thermal stabilization of the target RNA.

To gain insight into the prevalence of the 5′G_G/3′CAU bulge that Synucleozid binds throughout the human transcriptome, a database of secondary structural elements present in human miRNA hairpin precursors and highly expressed human RNAs with known structures was queried. The latter includes 5S rRNA, 16S rRNA, 23S rRNA, 7SL (signal recognition particle), RNase P RNA, U4/U6 snRNA, and 465 non-redundant tRNAs (2,459 total motifs). Among 7,436 motifs in miRNA hairpin precursors, 5′G_G/3′CAU only occurs twice, once in miR-1207 and once in miR-4310 (0.027%). The bulge only appears three times in highly expressed RNAs, each in a tRNA (0.12%). Further, various study have shown that typically small molecules must target a functional site in order to induce downstream biological effects²⁶. Many factors affect the biological response of molecular recognition of an RNA target, including target abundance and molecular recognition of a functional site²⁶⁻⁴¹. By analysis of these factors, the data support that targeting this 3D structure in SNCA IRE selectively is indeed achievable and to elicits a selective biological response.

### Biology of Synucleozid Embodiments of Formula II and their SAR

To further investigate molecular recognition at the small molecule level, a series of synucleozid A derivatives were synthesized and studied to determine structure and activity relationships (Fig. S10A). These compounds were designed to have improved blood-brain barrier (BBB) penetrance as defined by Central Nervous System Multiparameter Optimization (CNS MPO) scores⁴². In particular, the guanidyl groups were replaced with imidazolyl or cyano groups, while functionalities within the heterocycle as well as the amino group linking the two phenyl substituents were altered to study how changes in hydrogen bonding and stacking capacity affect molecular recognition (Fig. S10A).

Replacement of guanidyl groups with imidazolyl groups (SynucleoziD-2-SynucleoziD-5) largely increased compound CNS MPO scores without significantly affecting their avidity to the SNCA IRE as measured in the 2-AP fluorescent binding assay (Fig. S10B and Table S2). Despite the relatively small change in avidity, the cellular potency of all four derivatives was reduced compared to synucleozid A (i.e., Synucleozid of Fig S10A, with Im¹ and Im² as guanidyl. At 1 μM concentration, synucleozid A reduced α-synuclein levels by ˜67%. In contrast, the four imidazolyl derivatives only reduced α-synuclein levels by ˜40% at 5 μM. Replacement of the guanidyl groups with cyano (SynucleoziD-NC) ablated both binding avidity and its inhibitory effect on SNCA mRNA translation (Fig. S10C and Table S2).

### Study of Molecular Recognition of Synucleozid (Synucleozid A) to SNCA IRE Via ASO-Bind-Map

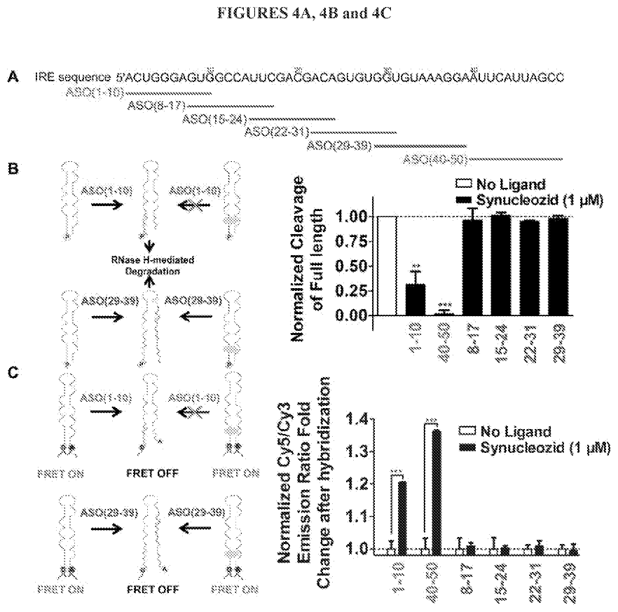

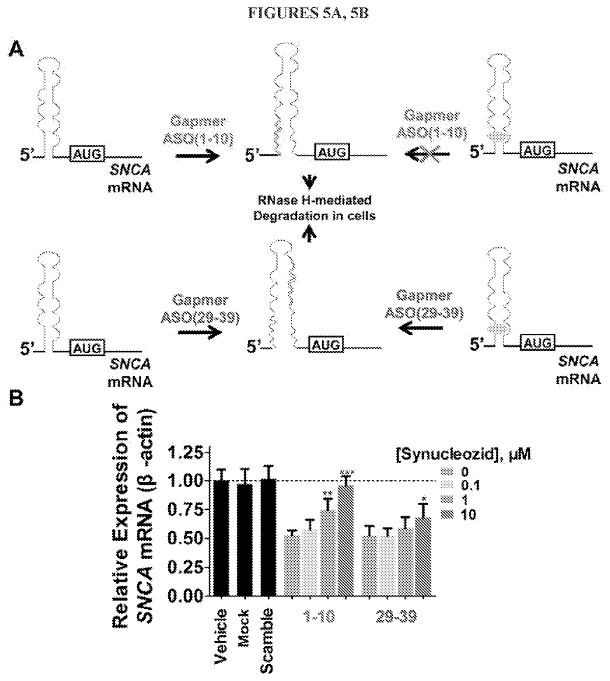

Traditionally, chemical mapping studies are used to identify ligand binding sites in vitro. However, compounds must be highly resident to detect their binding; that is, they must interact with the target site for sufficient time to inhibit an irreversible reaction with a chemical modification probe. Further confounding this analysis is that the sites that react with the modification reagent may not overlap with the ligand binding sites, leaving these sites invisible to detection⁴³. The ASO-Bind-Map (antisense oligonucleotide ligand binding site mapping) was developed to alleviate challenges associated with all three methods. Previously, the Williamson laboratory explored the folding pathways of large, highly structured RNAs using a series of ASOs⁴⁵⁻⁴⁶. Domains within the RNA that fold quickly into stable structures are largely inaccessible to ASO binding and hence cleavage while ones that did not fold or folded more slowly were subjected to ASO-mediated cleavage. This approach was adapted to profile the binding sites of small molecules to RNAs both in vitro and in cellulis. That is, since Synucleozid (synucleozid A) stabilizes the IRE structure, it should impede ASO binding and reduce RNase H cleavage at the binding site (FIGS. 4 and S11).

After designing and validating six tiling ASOs that bind throughout the SNCA IRE hairpin structure (FIG. 4A, Table S3), ASO-Bind-Map was implemented in two ways, using: (i) a ³²P-labeled IRE and analysis by gel electrophoresis (FIG. 4B); and (ii) a dually labeled IRE that is a fluorescent-based molecular beacon (FIG. 4C). In the first experiments, ³²P-labeled IRE was incubated 0.1, 1, and 10 μM of Synucleozid followed by addition of ASO and then RNase H. As expected, protection of the IRE from RNase H cleavage by Synucleozid was only observed with ASOs whose binding sites overlap with the A bulge, namely ASO (1-10) and ASO (40-50) (FIGS. 4B and S11).

In the molecular beacon assay, a model of the SNCA IRE was dually labeled on the 5′ and 3′ ends with Cy3 and Cy5, respectively, a fluorescence resonance energy transfer (FRET) pair. Upon hybridization of an ASO, the hairpin unfolds, and FRET is reduced. Again, if a small molecule is bound to a structure that overlaps with the sequence recognized by an ASO, the structure is stabilized, impeding ASO binding and the extent of FRET reduction slows. Each ASO was validated for reducing FRET in this system (Fig. S12). In agreement with the RNase H-mediated studies, Synucleozid was only able to reduce the extent of unfolding induced by ASO(1-10) and ASO(40-50), which bind sequences that overlap with the Synucleozid binding site, indicating specific binding to the A bulge (FIGS. 4C and S12).

Collectively, these three different assays, 2-AP-labeled RNA (including mutational studies). RNase H-mediated ASO-Bind-Map, and molecular beacon ASO-Bind-Map, all support binding of Synucleozid to the A bulge as designed.

To profile the binding of Synucleozid to the hairpin structure of the SNCA 5′ UTR in cells by ASO-Bind-MAP, ASO gapmers (2′-O-Methoxyelthyl (MOE) phosphorthioates) were used. Three oligonucleotides were studied: (i) the gapmer version of ASO(1-10), which overlaps with the Synucleozid binding site; (ii) the gapmer version of ASO(29-39), which does not overlap with the Synucleozid binding site; and (iii) a gapmer control ASO that does not share sequence complementarity with SNCA mRNA but has the same number and position of 2′MOE modifications and is the same length as ASO(1-10) and ASO(29-39) (Table S3).

Akin to in vitro studies, a gapmer of interest was transfected into SH-SY5Y cells in the presence and absence of Synucleozid (FIG. 5A). As expected, ASO(1-10) and ASO(29-39) (200 nM), but not the scrambled control ASO (200 nM), cleaved SNCA mRNA, reducing its levels by ˜50%, as measured by RT-qPCR. Addition of 0.1, 1, and 10 μM of Synucleozid to cells afforded dose-dependent inhibition of SNCA mRNA cleavage by ASO(1-10), which binds the sequence in and around the A bulge, with an EC₅₀ of 1 μM; no effect was observed with ASO(29-39) or the control ASO (FIG. 5B). These findings show direct target engagement in cells to further support that Synucleozid binds to the three-dimensional structure in and around the A bulge in the SNCA 5′ UTR structure. Cellular engagement of the target at the designed site is an important step to validate a compound's mode of action.

### Cellular Mechanism of Action of Synucleozid

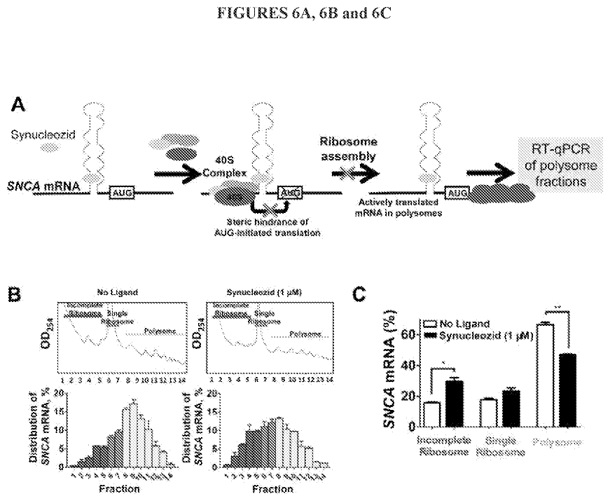

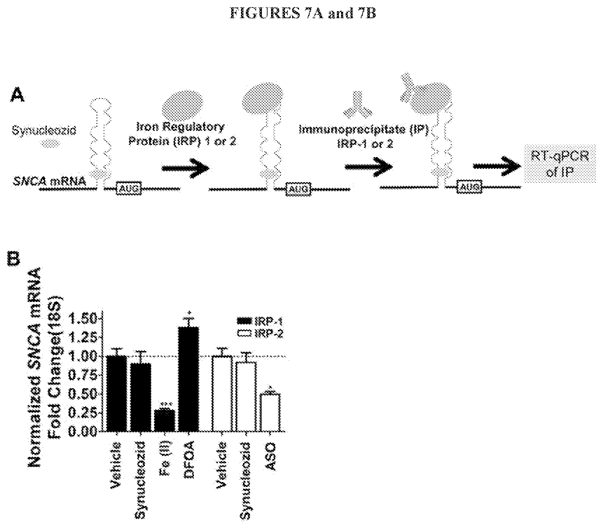

Three potential mechanisms of action through which Synucleozid could inhibit α-synuclein translation were investigated: (i) Synucleozid stabilizes the IRE hairpin structure and prevents its unfolding, blocking the pre-initiation ribosome complex from scanning through the IRE portion of the mRNA and obstructing the assembly of translationally competent ribosomal machinery (FIG. 6A); (ii) Synucleozid could increase expression of the iron response element binding protein (IRP) and facilitate IRP and SNCA IRE complex formation (FIG. 7A): IRP-IRE complex formation leads to translational inhibition²⁰⁻²¹; and (iii) Synucleozid binding to the IRE could increase the affinity between IRP and IRE, consequently repressing translation (FIG. 7A).

First investigated was whether Synucleozid affects ribosome assembly onto SNCA mRNA using polysome profiling. Polysome profiling is a powerful technique to study the association of mRNAs with ribosomes and to assess which mRNAs are undergoing active translation via their association in polysomes and the density of ribosomal loading⁴⁷. polysomes in the presence and absence of Synucleozid were collected and isolated through sucrose gradient (FIG. 6B, top). Fractions of the polysomes as a function of sucrose density gradient (e.g. the number of loaded ribosomes onto mRNAs) were collected, and the amount of SNCA mRNA relative to a control mRNA was measured by RT-qPCR (FIG. 6B, bottom).

Treatment with Synucleozid alters the distribution of SNCA mRNA between incomplete ribosomes (association with 40S or 60S subunits; fractions 1-5), single ribosomes (80S; fractions 5-7), and polysomes (fractions 8-14) (FIGS. 6B and 6C), but does not affect the association of ribosomes with a control RNA. In particular, Synucleozid decreases the amount of SNCA mRNA associated with active polysomes by ˜20% (p<0.05) with a concomitant increase in the amount associated with incomplete ribosomes (p<0.05) (FIG. 6C). Canonical translation of eukaryotic mRNAs is initialized by recruiting the 40S ribosomal subunit to the 5′ cap⁴⁸. The 40S and initiation factors form the pre-initiation complex that scans from 5′ end to AUG start codon by unfolding the 5′ UTR, followed by recruitment of 60S. The complete 80S ribosome machinery then elongates through the open reading frame. The observed significant increase of mRNA in fractions 1-5 shows that Synucleozid inhibits translation during the pre-initiation complex scanning but not the elongation stage. Collectively, these findings in conjunction with the in vitro results from ASO-Bind-Map support that Synucleozid inhibits ribosomal loading onto the SNCA mRNA by stabilizing the IRE and preventing its unfolding.

Following this demonstration of inhibition, a study of whether Synucleozid affects SNCA mRNA recognition by the two IRP isoforms, IRP-1 and IRP-2 (FIG. 7A) was conducted. IRP-1 is an abundant protein that not only serves as an iron-responsive protein but also as an aconitase to catalyze the conversion of citrate to isocitrate⁴⁹. IRP-2 is a less abundant form and differs from IRP-1 by a 73-amino acid insertion, removing its aconitase activity and facilitating its degradation in cells with low iron levels⁵⁰. To exclude the possibility that Synucleozid affects IRP expression, SH-SY5Y cells were treated with Synucleozid, and IRP abundance was measured by Western blotting. No change in the levels of either protein was observed (Fig. S13).

Next, IRP cellular complexes were isolated by immunoprecipitation (IP) and analyzed to assess if Synucleozid affects loading of IRPs onto the IRE of SNCA mRNA (FIG. 7A). A series of control experiments were completed for both IRP-1 and IRP-2 to ensure that differences in SNCA mRNA levels could be assayed (FIG. 7B). For IRP-1, treatment with iron (II) should decrease the amount of SNCA mRNA pulled down in the IP fractions, as iron (II) binds to IRP-1 and inhibits formation of the SNCA mRNA-IRP-1 complex, which was experimentally observed (FIG. 7B).

As expected, the amount of pulled down SNCA mRNA increased in the presence of the iron chelator deferoxamine (DFOA) (FIG. 7B). The IRP-2 expression is dependent on iron (II) concentration⁵¹. Therefore, as an experimental control, cells were treated with an ASO complementary to the IRP-2 binding site in the SNCA mRNA, which blocked IRP-2 binding and reduced the amount of immunoprecipitated SNCA mRNA (FIG. 7B). Importantly, Synucleozid did not affect the amount of SNCA mRNA associated with IRP-1 or IRP-2 in these carefully controlled IP experiments, consistent with in vitro displacement assays (FIGS. 7B and S14).

Collectively, these mechanistic investigations support a mechanism by which Synucleozid binds to the A bulge three-dimensional structure within the IRE hairpin of SNCA mRNA and inhibits the association of functional ribosomes to mRNA. (FIG. 6A).

### Evaluation of Synucleozid Selectivity

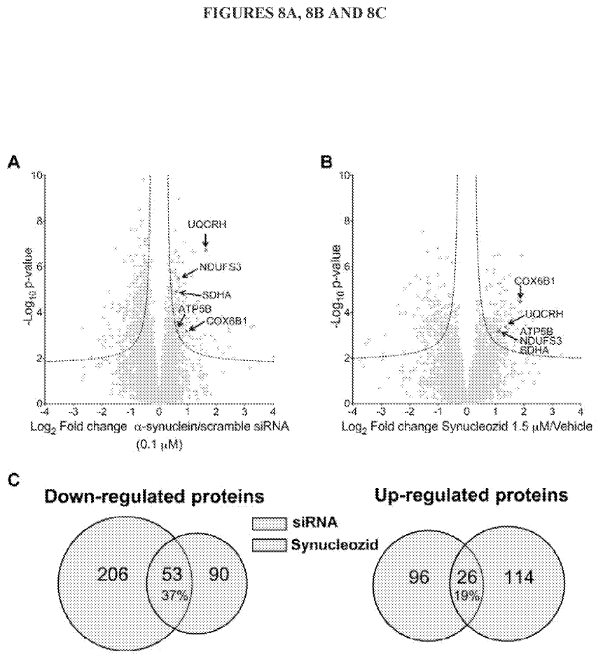

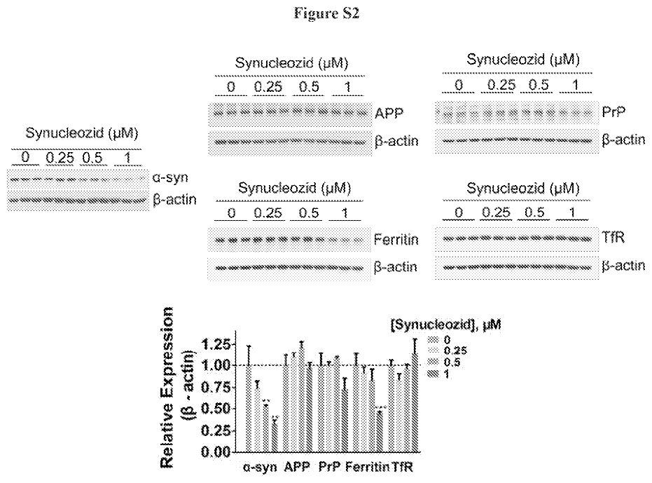

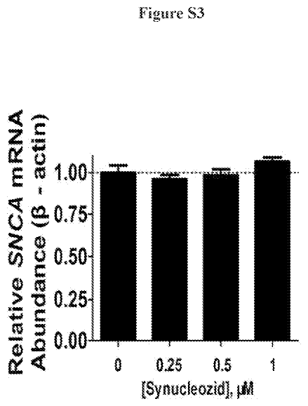

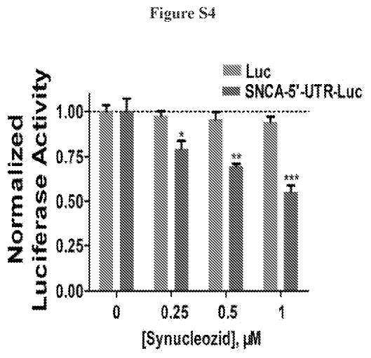

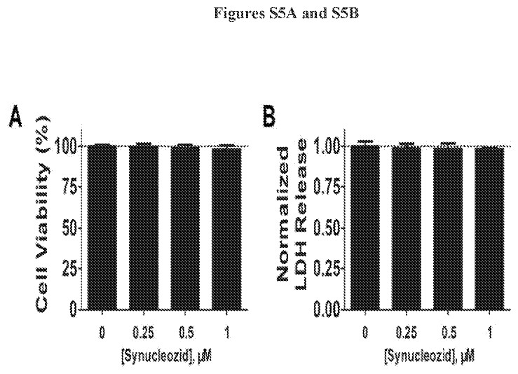

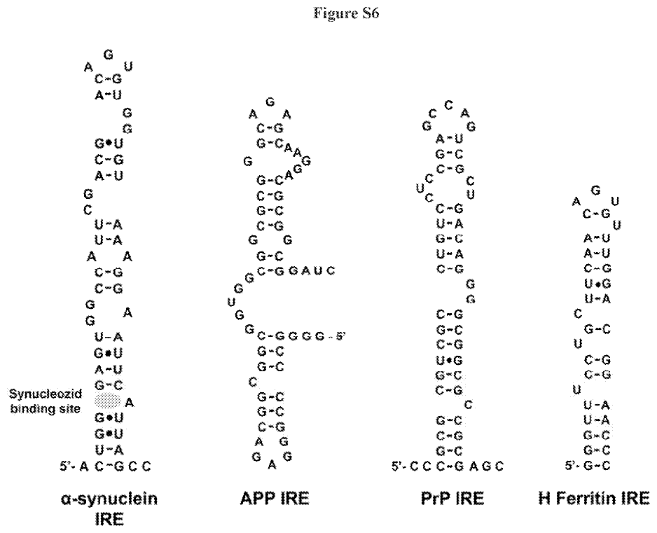

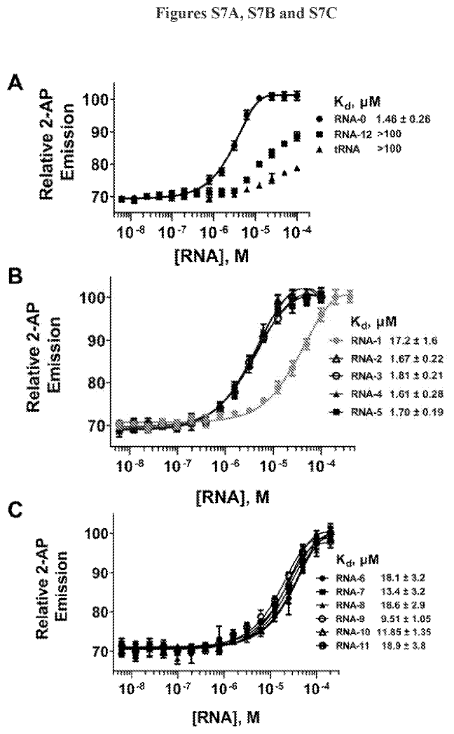

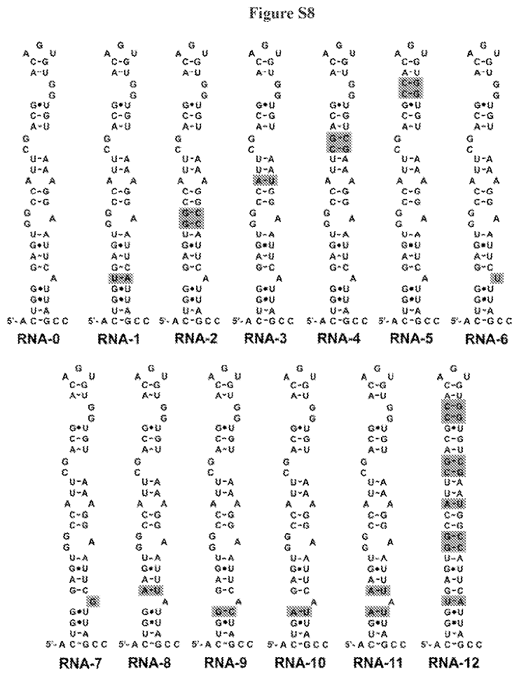

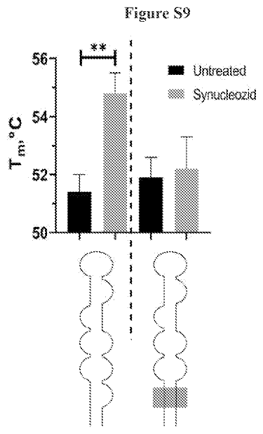

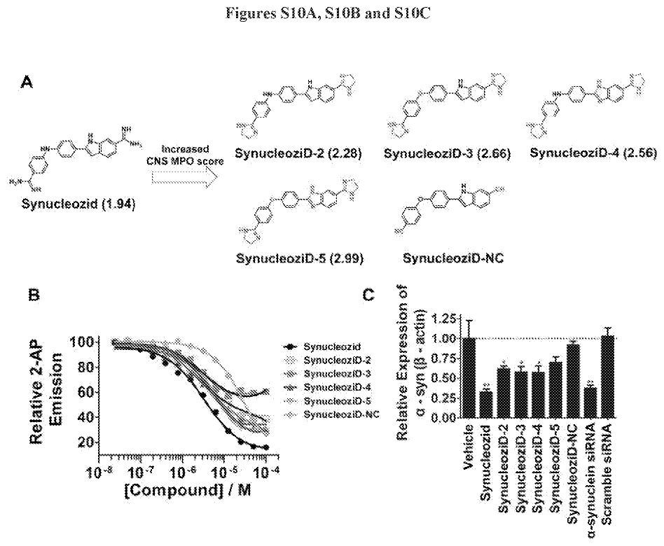

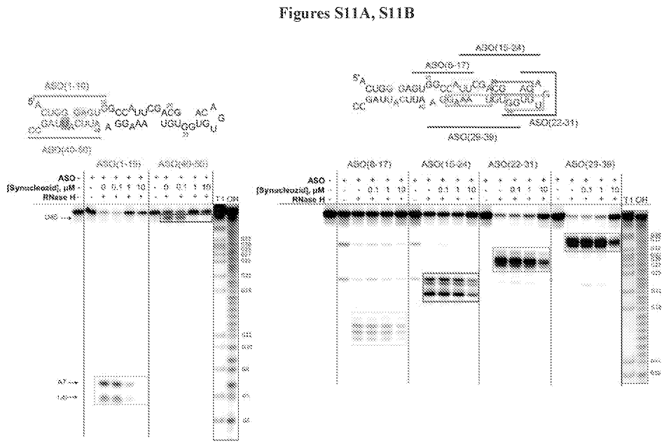

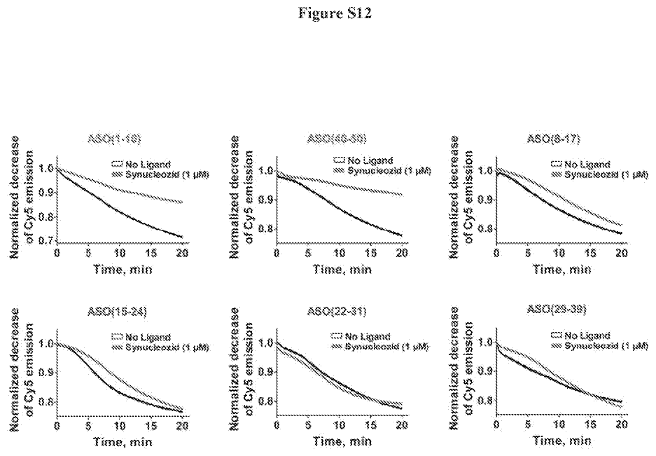

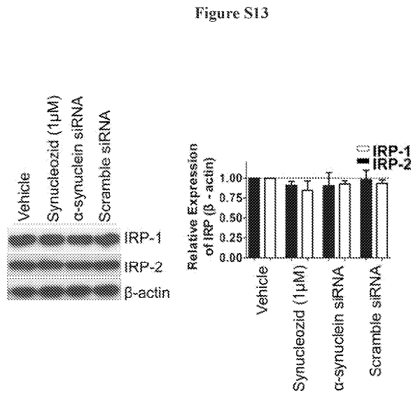

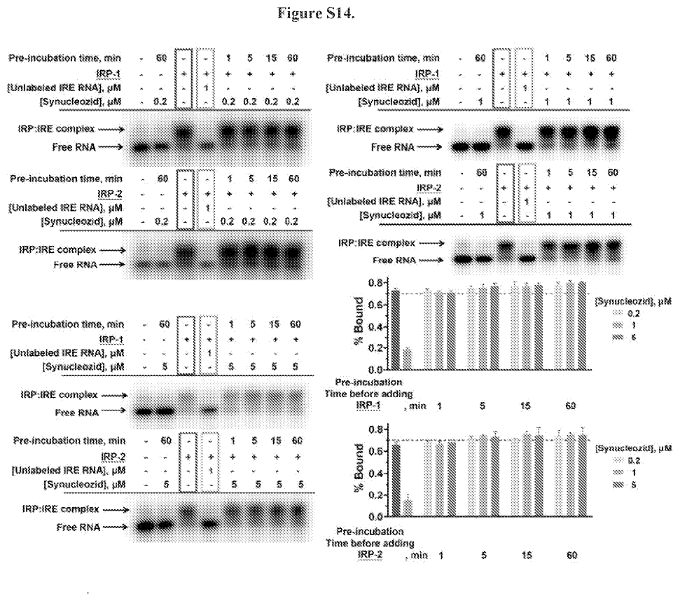

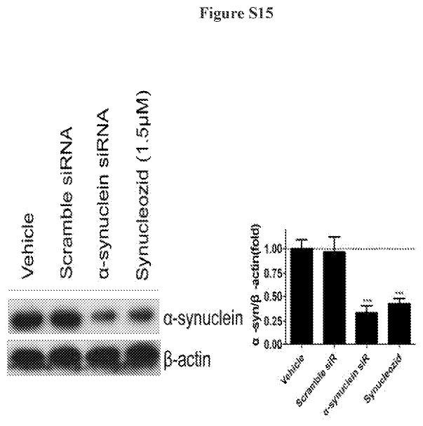

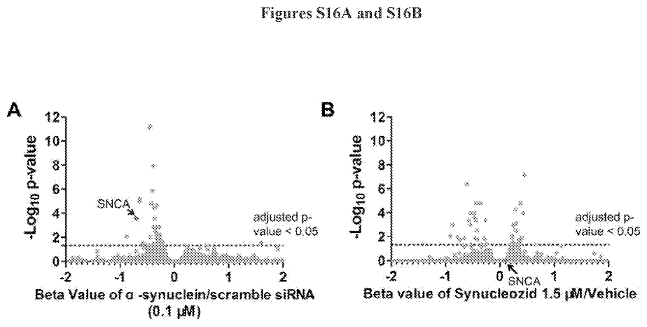

To assess the proteome-wide selectivity of Synucleozid for reducing α-synuclein protein levels, a series of studies were completed, comparing SH-SY5Y cells treated with: (i) Synucleozid; (ii) vehicle control; (iii) α-synuclein siRNA; and (iv) a scrambled control siRNA (Fig. S15). Among the 3300 proteins detected, 381 proteins (11%) were affected by α-synuclein siRNA treatment (relative to scrambled control siRNA-treated cells), while 283 (8%) of the proteins were significantly (adjusted p-value <0.01) affected with the cell-permeable small molecule Synucleozid (relative to vehicle-treated cells) (FIGS. 8A and 8B: a spreadsheet of full proteomics analysis is provided in Supporting Information). The siRNA downregulated 259 proteins (7.9% of all proteins) while Synucleozid downregulated 143 (4.3% of all proteins), of which 53 overlap. The number of proteins with increased expression for the siRNA and Synucleozid are similar, 122 (3.7% of all proteins) and 140 (4.2% of all proteins), respectively, with 26 common proteins in the two datasets.

These overlapping proteins could be involved in α-synuclein-related pathways. For example, previous studies indicated that α-synuclein impairs the mitochondrial complex in the brain⁵²⁻⁵³; thus, inhibition of α-synuclein synthesis could recover this phenotype. Indeed, down-regulation of α-synuclein by compound and siRNA treatment caused a common up-regulation of proteins involved in the oxidative phosphorylation pathway, such as ATP5B, NDUFS3, COX6B1, SDHA, and UQCRH (FIGS. 8A and 8B). Other proteins encoded by mRNAs with IREs were searched in proteomics data (n=16; Supporting Information dataset), and four were found which were expressed at measurable levels, ACO2. NDUFS1, CDC42BPB, and FTH1 (ferritin). Only FTH1 was affected by Synucleozid treatment, as expected from the cellular studies presented in FIG. 2. This shows that Synucleozid (synucleozid A) demonstrates proteome-wide selectivity⁵⁴.

In complementary studies, the transcriptome-wide effect of Synucleozid treatment were examined using RNA-Seq. Differentially expressed genes were identified using quantification and analyses from Kallisto and Sleuth packages in R²⁹. Very few changes were observed upon Synucleozid (19979/20034 genes were unchanged; 99.7%) or siRNA treatment (19279/19329 genes were unchanged: 99.7%), suggesting limited off-target effects for either modality (Fig. S16).

### Mechanism of Action and Medical Treatment

In certain embodiments, the invention is directed to methods of inhibiting, suppressing and/or managing biolevels of α-synuclein mRNA. The compounds of Formulas I and II of the invention for use in the methods disclosed herein bind to IRE active site of mRNA for α-synuclein.

Embodiments of the compounds applied in methods of the invention and their pharmaceutical compositions are capable of acting as “inhibitors”, suppressors and or modulators of mRNA for α-synuclein which means that they are capable of blocking, suppressing or reducing the expression of α-synuclein. An inhibitor can act with competitive, uncompetitive, or noncompetitive inhibition. An inhibitor can bind reversibly or irreversibly.

The compounds useful for methods of the invention and their pharmaceutical compositions function as therapeutic agents in that they are capable of preventing, ameliorating, modifying and/or affecting a disorder or condition. The characterization of such compounds as therapeutic agents means that, in a statistical sample, the compounds reduce the occurrence of the disorder or condition in the treated sample relative to an untreated control sample, or delays the onset or reduces the severity of one or more symptoms of the disorder or condition relative to the untreated control sample.

The ability to prevent, ameliorate, modify and/or affect in relation to a condition, such as a local recurrence (e.g., pain), a disease known as a synucleinopathic disease such as but not limited to Parkinson's disease, a syndrome complex such as nerve impairment or any other medical condition, is well understood in the art, and includes administration of a composition which reduces the frequency of, or delays the onset of, symptoms of a medical condition in a subject relative to a subject which does not receive the composition. Thus, prevention of synucleinopathic disease includes, for example, reducing the number of symptoms in a treated population versus an untreated control population, e.g., by a statistically and/or clinically significant amount. Associated hallucinogenic issues may also be ameliorated and/or minimized and include, for example, reducing the magnitude of, or alternatively delaying, untoward nerve sensations experienced by subjects in a treated population versus an untreated control population.

The compounds of the invention and their pharmaceutical compositions are capable of functioning prophylactically and/or therapeutically and include administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the tem “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated, e.g., at a reasonable benefit/risk ratio applicable to any medical treatment.

### Administration

Compounds of the invention and their pharmaceutical compositions prepared as described herein can be administered in various forms, depending on the disorder to be treated and the age, condition, and body weight of the patient, as is well known in the art. As is consistent, recommended and required by medical authorities and the governmental registration authority for pharmaceuticals, administration is ultimately provided under the guidance and prescription of an attending physician whose wisdom, experience and knowledge control patient treatment.

For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route or other similar transmucosal route, they may be formulated as drops or ointments.

These formulations for administration orally or by a transmucosal route can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the gender of the patient, the nature and severity of the disorder to be treated or prevented, the route of administration and the form of the drug, in general, a daily dosage of from 0.0001 to 2000 mg, preferably 0.001 to 1000 mg, more preferably 0.001 to 500 mg, especially more preferably 0.001 to 250 mg, most preferably 0.001 to 150 mg of the compound is recommended for an adult human patient, and this may be administered in a single dose or in divided doses. Alternatively, a daily dose can be given according to body weight such as 1 nanogram/kg (ng/kg) to 200 mg/kg, preferably 10 ng/kg to 100 mg/kg, more preferably 10 ng/kg to 10 mg/kg, most preferably 10 ng/kg to 1 mg/kg. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those excipients, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Pharmaceutical Compositions Incorporating Compounds of Formulas I and II

The pharmaceutical compositions of the invention incorporate embodiments of a compounds of Formulas I and II useful for methods of the invention and a pharmaceutically acceptable carrier. The compositions and their pharmaceutical compositions can be administered orally, topically, parenterally, by inhalation or spray or rectally in dosage unit formulations. The term parenteral is described in detail below. The nature of the pharmaceutical carrier and the dose of the compounds of Formulas I and II depend upon the route of administration chosen, the effective dose for such a route and the wisdom and experience of the attending physician.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose: (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil: (10) glycols, such as propylene glycol; (11) polyols, such as glycerin, sorbitol, mannitol, and polyethylene glycol; (12) esters, such as ethyl oleate and ethyl laurate: (13) agar: (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia:
(3) humectants, such as glycerol;
(4) disintegrating agents, such as agar-agar, calcium carbonate, potato or tapioca starch, alginic acid, certain silicates, and sodium carbonate:
(5) solution retarding agents, such as paraffin;
(6) absorption accelerators, such as quaternary ammonium compounds;
(7) wetting agents, such as, for example, acetyl alcohol and glycerol monostearate;
(8) absorbents, such as kaolin and bentonite clay;
(9) lubricants, such a talc, calcium stearate, magnesium stearate, solid polyethylene glycols, sodium lauryl sulfate, and mixtures thereof; and
(10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound useful for application of methods of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound useful for practice of methods of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) useful for application of the methods of the invention may be administered to humans and other animals for therapy by any suitable route of administration, including orally, nasally, as by, for example, a spray, rectally, intravaginally, parenterally, intracistemally, and topically, as by powders, ointments or drops, including buccally and sublingually.

Regardless of the route of administration selected, the compound(s) useful for application of methods of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) useful for application of methods of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound useful for application of methods of the invention in a pharmaceutically acceptable mixture will vary depending on several factors, including the dosage of the compound to be administered, the pharmacokinetic characteristics of the compound(s) employed, and the route of administration.

In general, the compositions useful for application of methods of this invention may be provided in an aqueous solution containing about 0.1-10% w/v of a compound disclosed herein, among other substances, for parenteral administration. Typical dose ranges are those given above and may preferably be from about 0.001 to about 500 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

### Materials & Methods

Detailed information for all experimental methods and materials, including synthetic methods and compound characterization, are provided in the following sections.

Statistical analysis. Data are presented as means±SD from at least three independent biological replicates. Statistical significance between experimental groups was analyzed either by two-tailed Student t test or one-way ANOVA followed by Bonferroni's multiple comparison test. In all cases, p values of less than 0.05 were considered to be statistically significant.

Cell culture. Provided in the following sections.

Quantitative Real-Time PCR (RT-qPCR). After completion of treatment, total RNA was extracted from cells (RNeasy mini kit. Qiagen) and cDNA was synthesized (Superscript II, Invitrogen) according to the manufacturer's instructions. Quantitative RT-PCR was performed in triplicate using iTaq Universal SYBR Green Supermix (Bio-Rad) with Applied Biosystems 7500 Real-Time PCR system to assess the relative SNCA mRNA levels. Please see the Supporting Information for additional details including primer sequences.

Western blotting. Protein expression of α-synuclein, APP, ferritin and TfR were analyzed in SH-SY5Y cells, while the level of PrP was tested in Neuro-2A cells after compound or vehicle treatment. To improve immunodetection of endogenous α-synuclein, membranes used to probe for endogenous α-synuclein were mildly fixed with 0.4% paraformaldehyde for 30 min at room temperature prior to the blocking step⁷⁷. Additional details are provided in the Supporting Information.

Cell death assay. The cytoprotective effect of Synucleozid was tested against α-synuclein PFFs. Human α-synuclein was expressed⁷⁸ and fibrillization induced as previously described⁷⁹. SH-SY5Y cells were pretreated with Synucleozid (0.25 μM to 1 μM) for 24 h and challenged with 50 μg/mL of α-synuclein PFFs for an additional 48 h in the presence of Synucleozid. Cell death was measured using LDH (Lactate Dehydrogenase) Cytotoxicity Detection Kit (Takara) according to the manufacturer's instructions and plotted either as optical density value or as percentage changes in comparison to respective controls. Monomeric α-synuclein (50 μg/mL) challenge was used as negative control for PFF cytotoxicity. Additional details are provided in the Supporting Information.

To assess if Synucleozid has a cytotoxic effect, SH-SY5Y cells were treated with 0.25 μM to 1 μM for 48 h, and cell viability and cytotoxicity were measured using CellTiter 96 Aqueous One Solution Cell Proliferation Assay (MTS) according to the manufacturers' instructions.

### Synthetic Methods

Compounds 1(1), 2(1), SynucleoziD-2(2), SynucleoziD-3(3), SynucleoziD-5(3), SynucleoziD-NC(3), 5(4) were synthesized according to reported procedures. Compounds 3, 4 and Synucleozid were obtained from the National Cancer Institute (NCI). Deferoxamine was obtained from Sigma-Aldrich.

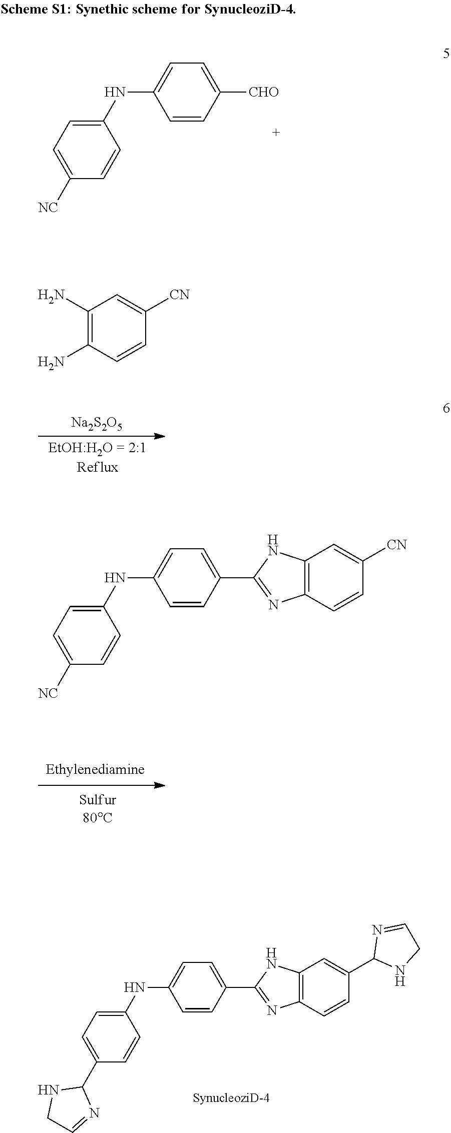

### Synthesis of SynucleoziD-4

A solution of 5 (180 mg, 0.81 mmol), 3,4-Diaminobenzonitrile (110 mg, 0.83 mmol) and Na₂S₂O₅ (175 mg, 1.68 mmol) in ethanol: water=2:1 was stirred at reflux for 4 h. The reaction mixture was cooled to room temperature and concentrated in vacuo. The residue was extracted by ethyl acetate (EA) from water, washed with brine, dried over anhydrous Na₂SO₄ and concentrated in vacuo. The residue was purified by column chromatography (EA:Hex=1:2) to give a yellow solid (211.5 mg, 0.63 mmol, 78%).

¹H NMR (400 MHz, DMSO-d⁶) δ 9.41 (s, 1H), 8.17-8.15 (m, 3H), 7.79-7.77 (dd, J=8.36 Hz, 0.56 Hz, 1H), 7.72-7.70 (m, 2H), 7.69-7.66 (dd. J=8.36 Hz, 0.68 Hz, 1H), 7.39-7.37 (d, J=8.84 Hz, 2H), 7.29-7.27 (d, J=8.88 Hz, 2H). ¹³C NMR (100 MHz, DMSO-d⁶) δ 158.5, 158.1, 153.7, 146.4, 144.4, 139.9, 137.5, 133.7, 128.8, 126.4, 119.7, 119.6, 117.9, 116.6, 115.2, 104.7, 101.1. HRMS: (ESI) calcd for C₂₁H₁₄N₅ [M+H]⁺: 336.1244. found: 336.1238.

SynucleoziD-4: To a solution of 6 (35 mg, 0.10 mmol) in ethylenediamine (2 mL) at 80° C. was added sulfur (13 mg, 0.40 mmol). The mixture was stirred at 80° C. for 4 h. The reaction mixture was cooled to room temperature and concentrated in vacuo. The residue was re-dissolved in methanol and the solid was filtered off. The filtrate was concentrated in vacuo and the residue was purified by HPLC to afford SynucleoziD-4 as a yellow solid (5.1 mg, 0.012 mmol, 12.1%).

¹H NMR (400 MHz, CD₃OD) δ 8.22 (m, 1H), 8.16-8.14 (d, J=8.88 Hz, 2H), 7.86-7.79 (m, 4H), 7.46-7.44 (d, J=8.92 Hz, 2H), 7.37-7.34 (d, J=9 Hz, 2H), 4.15 (s, 4H), 4.07 (s, 4H).

¹³C NMR (100 MHz, CD₃OD) δ 168.1, 167.0, 156.3, 150.2, 146.4, 139.3, 138.9, 131.3, 130.2, 124.5, 121.3, 120.0, 118.4, 117.5, 117.0, 115.9, 114.0, 46.0, 45.7. HRMS: (ESI) calcd for C₂₅H₂₄N₇[M+H]˜: 422.2093. found: 422.2061.

### Other Methods

Modeling IRE secondary structure. The SNCA IRE structure model was manually constructed to maximize its similarity to the human ferritin IRE model (accessed from the Rfam database; Rfam accession #RF00037) generated from comparative sequence and structure analysis. The SNCA IRE is longer than the ferritin IRE sequence; thus, the basal stem of the SNCA IRE is based upon free energy minimization (using the program RNAfold) (5).

To check for conservation of the SNCA IRE model structure, an alignment of 55 homologous sequences (identified from a BLASTn search of the RefSeq RNA database) was generated using MAFFT (MAFFT-G-INS-I; Fig. S1, left panel). The SNCA IRE structure model was annotated with base pair conservation data and nucleotides that show evidence of structure-preserving mutations were identified manually (Fig. S1, right panel).

Cell culture. Human neuroblastoma SH-SY5Y cells (ATCC, Manassas, Va., USA) were cultured in Dulbecco's Modified Eagle's Medium/F-12 1:1 mix medium (DMEM/F-12, GE Healthcare, Chicago, Ill., USA) supplemented with 10% FBS (fetal bovine serum, Atlanta Biologicals, Flowery Branch, GA, USA). Human embryonic kidney HEK293T cells (ATCC) and mouse neuroblastoma Neuro-2A cells (ATCC) were cultured in DMEM supplemented with 10% FBS. All cells in culture were maintained in a humidified incubator at 37° C. in 5% CO₂. For the experiments, cells were seeded in 6, 12 or 24-well plates until reaching about 60-70% confluency, at which time the culture medium was replaced with fresh media containing either vehicle (DMSO, volume of DMSO is the same as compounds) or compounds for 48 h.

Quantitative Real-Time PCR (RT-qPCR). After completion of treatment, total RNA was extracted from cells (RNeasy mini kit from Qiagen, Germantown, Md., USA) and cDNA was synthesized (Superscript ii from Invitrogen, Carlsbad, Calif., USA) according to the manufacturer's instructions. Quantitative RT-PCR was performed in triplicate using iTaq Universal SYBR Green Supermix (Bio-Rad, Hercules, Calif., USA) with Applied Biosystems 7500 Real-Time PCR system to assess the relative SNCA mRNA levels (forward 5′-ACCAAACAGGGTGTGGCAGAAG-3′; reverse 5′-CTTGCTCTITGGTCTTCT CAGCC-3′). β-Actin (forward 5′-CATGTACGTTGCTATCCAGGC-3′; reverse 5′-CTCCTTAATGTCACG CACGAT-3′) was used as the endogenous reference gene for normalization, and the relative levels of SNCA mRNA (ΔΔCt value) were expressed as fold changes in comparison to the untreated control.

Western blotting. Protein expression of α-synuclein, amyloid precursor protein (APP), Ferritin and transferrin receptor were analyzed in SH-SY5Y neuroblastoma cells, while the level of prion protein (PrP) was tested in Neuro-2A cells after compound, vehicle or siRNAs treatment. Cells were gently washed twice with ice-cold PBS and lysed in 2% SDS lysis buffer containing protease (539137, Millipore, Burlington, Mass., USA) and phosphatase inhibitor cocktails (P5726, Sigma, St. Louis, Mo.), followed by brief sonication. After quantifying protein concentration using BCA Protein assay kit (Thermo Fisher Scientific, Waltham, Mass., USA), equal amount of protein from each sample was separated in 4-20% SDS-PAGE (GenScript, Piscataway, N.J., USA) and transferred onto polyvinylidene difluoride (PVDF) membranes. To improve immunodetection of endogenous α-synuclein, membranes used to probe for endogenous α-synuclein were mildly fixed with 0.4% paraformaldehyde for 30 min at room temperature prior to the blocking step (6). Non-specific binding was blocked with 5% non-fat dry milk for 1 h at room temperature, and membranes were probed overnight with primary antibodies specific for α-synuclein (1:1000, 610787 from BD Transduction Laboratories), APP (1:2000, ab32136 from Abcam), PrP (1:2000, ab52604 from Abcam), ferritin (1:2000, ab75973 from Abeam), and transferrin receptor (1:2000, ab84036 from Abeam). β-Actin (1:10000, A5441 from Sigma) was used as a protein loading control. After six 10-minute washes with TBST (Tris-buffered saline, 0.5% Tween 20), membranes were incubated in horseradish peroxidase (HRP)-conjugated secondary antibodies (1:2000 for α-synuclein, 1:5000 for APP, PrP, ferritin and transferrin receptor, 1:20000 for β-Actin) for 1 h at room temperature and washed six times with TBST. Immunocomplex signals of each protein were then detected using enhanced chemiluminescence detection system (ECL, PerkinElmer, Waltham, Mass., USA). Abundance of protein in each sample was determined based on band intensity from ImageJ analysis, then normalized to β-Actin bands, and expressed as fold changes compared with vehicle treated control cells.

Cell death assay. The cytoprotective effect of Synucleozid was tested against α-synuclein preformed fibrils. Human α-synuclein protein was expressed from plasmid pT7-7 in Escherichia coli BL21(DE3) strain (Invitrogen Inc.) and dissolved in PBS at a final concentration of 5 mg/mL (7). To induce fibrillization of monomertic α-synuclein, the solution was subjected to shaking at 1000 rpm at 37° C. for 7 days on a thermomixer C (Eppendorf), and formation of fibrillar α-synuclein was monitored and confirmed by thioflavin-T assay (8). SH-SY5Y cells were pre-treated with Synucleozid (0.25, 0.5 and 1 μM) for 24 h and challenged with 50 μg/mL of α-synuclein pre-formed fibrils (PFFs) for an additional 48 h in the presence of Synucleozid. Cell death was measured using LDH (Lactate Dehydrogenase) Cytotoxicity Detection Kit (Takara) according to the manufacturer's instructions and plotted either as optical density value or as percentage changes in comparison to respective controls. Monomeric α-synuclein (50 μg/mL) challenge was used as negative control for PFFs cytotoxicity.

To assess if Synucleozid has a cytotoxic effect, SH-SY5Y cells were treated with 0.25, 0.5 and 1 μM for 48 h, and cell viability and cytotoxicity were measured using CellTiter 96 Aqueous One Solution Cell Proliferation Assay (MTS) and LDH release assay, respectively, according to the manufacturers' instructions.

Plasmid constructs and establishment of reporter gene overexpressing cells. In-Fusion PCR cloning system (Takara Bio, Mountain View, Calif., USA) was used for all plasmid constructions according to the manufacturer's instructions. All plasmids generated in this study were confirmed by sequencing to ensure that there were no unwanted mutations in the inserts (See Table S5 for sequences of primers used to generate plasmid constructs).

### Cloning of Firefly Luciferase Reporter Constructs

To generate reporter gene constructs containing the 5′ UTR of human SNCA or human amyloid precursor protein (APP), the following steps were followed:

pIRES-Luc-EGFP-puro: The firefly luciferase open reading frame (ORF) was amplified with primers 1 and 2 using pmirGLO-dual-luciferase (Promega, E1330) as a template. The PCR product was then fused with linearized pIRES-EGFP-puro (Addgene plasmid #45567) digested with XhoI and SacI, placing the luciferase ORF upstream of the IRES sequence.

pCDH-α-syn-5′-UTR-Luc-EGFP-puro and pCDH-APP-5′-UTR-Luc-EGFP-puro: The 5′ UTR followed by several bases of ORF of human SNCA (primers 3 and 4), and that of human amyloid precursor protein (primers 5 and 6) were amplified from human cDNA library and then fused with linearized pIRES-Luc-EGFP-puro digested with XhoI. Subsequently, deletion mutations were made to remove the ORF bases of human SNCA (using primers 7 and 8) and human amyloid precursor protein (using primers 9 and 10). Finally, the PCR product containing 5′ UTR of human SNCA (primers 11 and 12) or human amyloid precursor protein (primers 13 and 14) with firefly luciferase ORF was fused with linearized pCDH-CB-IRES-copGFP-T2A-Puro (Addgene plasmid #72299) digested with BamHI and NotI.

Reporter gene construct containing the 5′ UTR of human prion protein was generated as follows:

pCDH-Luc-EGFP-puro: The firefly luciferase open reading frame (ORF) was amplified with primers 15 and 16 using pmirGLO-dual-luciferase (Promega, E1330) as a template. The PCR product was then fused with linearized pCDH-CB-IRES-copGFP-T2A-Puro digested with BamHI and NotI, placing the luciferase ORF upstream of IRES sequence.

pCDH-PrP-5′-UTR-Luc-EGFP-puro: Two complementary oligonucleotides (primers 17 and 18, 100 μM each) were annealed by using T4 PNK (NEB, M0201S) (parameters: 37° C. for 30 min; 95° C. for 5 min; ramp down to 25° C. at 0.1PC per second) to form the double-stranded 5′ UTR of PrP variant 2. Then the double-stranded PrP 5′UTR oligonucleotide was fused with linearized pCDH-CB-IRES-copGFP-T2A-Puro digested with BamHI, upstream of the firefly luciferase ORF.

To generate a reporter gene construct containing 5′ UTR of human ferritin (pCDH-Ferritin-5′-UTR-Luc-EGFP-puro), the 5′ UTR of ferritin (primers 19 and 20) was amplified from cDNA library and fused with linearized pCDH-Luc-EGFP-puro digested with BamHI, placing the insert upstream of the firefly luciferase ORF.

To produce lentiviral vectors expressing these plasmids, HEK293T cells were co-transfected with three plasmids (lentiviral construct overexpressing each target sequence, packaging plasmid; psPAX2 and envelope plasmid; pMD2.G). After 48 h of transfection, virus-containing supernatants were collected and used to transduce SH-SY5Y cells. SH-SY5Y cells with stable integration of each construct (GFP positive) were selected using 2 μg/mL of puromycin (Sigma) after 48 h of virus transduction.

Luciferase assay. The effect of Synucleozid treatment on translation from various IRE containing transcripts was determined using luciferase reporter assay. SH-SY5Y cells stably expressing 5′ UTR were treated with Synucleozid for 48 h, washed with PBS and lysed with Passive Lysis Buffer for 15 min at room temperature. Luciferase reporter activity in each lysate was monitored using the dual luciferase reporter assay system kit (Promega. Madison, Wis., USA) according to the manufacturer's instructions. Luminescence from firefly luciferase reaction was measured using a microplate luminometer (Wallac 1420, Perkin Elmer) at 485 nm excitation and 535 nm emission wavelengths. Firefly luciferase reaction was then quenched by adding Stop & Glo reagent, and the internal fluorescence from GFP measured at the same setting was used to normalize luciferase expression. Luciferase reporter activity was expressed as percent change compared to the respective vehicle-treated control cells. In vitro translation assay. The mRNA template for in vitro translational assays was transcribed from the pIRES-Luc-EGFP-puro and pCDH-α-syn-5′-UTR-Luc-EGFP-puro plasmids by using an in vitro transcription system (RiboMax, Promega) with forward primer: 5′-GGCCGGATACTAATACGACTCACTATAGGCT GCGGAATTGTACC-CG-3′ and reverse primer: 5′-GAGGGGCGGATCAATT-3′. In vitro translation reactions (50 μL in total for each condition) with the Flexi Rabbit Reticulocyte Lysate System (Promega) were programmed with 2 μg mRNAs. The mRNA was folded in buffer containing KCl (100 mM) by heating at 65° C. for 10 min followed by slow cooling to room temperature. Then RNA solution was added with reticulocyte lysate, amino acid mixtures, and RNasin ribonuclease inhibitor (Promega) to 50 μL (manufacturer's protocol). Translation proceeded at 30° C. for 90 min, and then luminescence was measured with a Biomek FLx800 plate reader (1.0 s integration time).

2-AP emission assay. One μM 2-AP labeled SNCA IRE RNA was folded by heating to 65° C. for 5 min in 1× Assay Buffer and slowly cooling to room temperature. Bovine serum albumin (BSA) was then added to the RNA solution to 40 μg/mL. After adding Synucleozid to RNA solution at a final concentration of RNA of 100 μM, serial dilutions were prepared using 1× Assay Buffer including BSA supplemented with folded one μM 2-AP labeled SNCA IRE RNA. The solutions were incubated at room temperature for 30 min in dark and transferred into wells of black 384-well half area plates (Greiner). Fluorescence intensity was measured at room temperature with Tecan plate reader (Gain: 100, Integration time: 40 μs). Fluorescence excitation and emission wavelengths were set at 310 and 380 nm. The change of 2-AP fluorescence intensity as a function of Synucleozid concentration was fit to Eq. (1):

I=I₀+0.5Δε{([FL]₀+[RNA]₀+EC₅₀)−(([FL]₀+[RNA]₀+EC₅₀)²−4[FL]₀[RNA]₀)^{0.5}} Eq. (1)

I and I₀ are the observed fluorescence intensity in the presence and absence of 2-AP RNA. Δε is the difference between the fluorescence intensity in the absence and presence of infinite 2-AP RNA. [FL]₀ and [RNA]₀ are the concentrations of Synucleozid and 2-AP RNA, respectively. EC₅₀ was calculated from Eq. (1). Competitive binding assay was performed by incubating 2-AP labeled SNCA IRE RNA with 2.7 μM Synucleozid (EC₅₀ determined in the assay above) and increasing concentrations of competitive RNAs (RNA-0 to RNA-12 and tRNA). The change of fluorescence intensity was fit to Eq. (2):

$$θ = \frac{1}{2 [ RNA ]} [ E {C}_{50} + \frac{{EC}_{50}}{{K}_{d}} [ {C}_{t} ] + [ Synucleozid ] + [ R N A ] ] - {\{ {( {EC}_{50} + \frac{E {C}_{50}}{{K}_{d}} + [ {C}_{t} ] + [ Synucleozid ] + [ RNA ] )}^{2} - 4 [ Synucleozid ] [ RNA ] \}}^{0.5} + A \qquad Eq . ( 2 )$$

θ is the percentage of 2-AP RNA bound. [RNA] is the concentration of 2-AP RNA. EC₅₀ is the EC₅₀ of Synucleozid determined in the assay above. [Synucleozid] is the concentration of Synucleozid. Cc is the concentration of competitive RNA. K_{d} is competitive dissociation constant and A is a constant.

Thermal melting experiment. Thermal stability of RNAs was analyzed by optical melting experiments with and without Synucleozid. The RNA was heated to 65° C. and slowly cooled to room temperature. The samples were then cooled to 20° C. upon addition of Synucleozid and then heated to 85° C. at a rate of 1° C. per minute. The absorbance of solution was monitored at 260 nm by a Beckman Coulter DU 800 Spectrophotometer. Background of buffer or buffer plus compound was subtracted from the signal. Resulted melting curves were fitted with MeltWin v3.5 to determine ΔG°, ΔH°, ΔS°, and Tₘ. The melting temperature was compared to the first derivative value from the raw data for similarity.

In vitro mapping of SNCA IRE structure. The SNCA IRE hairpin RNA was 5′-end labeled with [γ-³²P] ATP and T4 kinase as previously described⁵. Approximately 5 pmoles of ²P-labeled RNA per 20 μL reaction was folded in 1× Assay Buffer (8 mM Na₂HPO₄. pH 7.0, 190 mM NaCl, and 1 mM EDTA) by heating at 65° C. for 5 min and then slowly cooling to mom temperature. Varying concentrations of RNase T1 (1:2 serial dilutions affording final concentrations from 3 unit/μL to 0.047 unit/μL) were then added, and the samples were incubated at room temperature for 20 min.

Two ladders were generated for comparison and determining the positions of cleavage. All guanosine (G) residues were identified by RNase T1 cleavage under denaturing condition. Briefly, the RNA was denatured in 1×RNA Sequencing Buffer (20 mM sodium citrate, pH 5.0, 1 mM EDTA and 7 M Urea) at 60° C. for 10 min. After cooling to room temperature. RNase T1 was added at varying concentrations up to a final concentration of 3 unit/μL, followed by a 20 min incubation at room temperature. The hydrolysis ladder was prepared by incubating the RNA in 1×RNA Hydrolysis Buffer (50 mM sodium bicarbonate, pH 9.4 and 1 mM EDTA) at 95° C. for 2 min.

All reactions were quenched by adding an equal volume of 2× Loading Buffer (95% formamide, 5 mM EDTA, and 0.025% (w/v) bromophenol blue and xylene cyanol FF). Fragments were separated on a denaturing 15% polyacrylamide gel (PAGE), imaged using a Bio-Rad PMI phosphorimager and analyzed and quantified by Bio-Rad's QuantityOne software.

RNase H-mediated ASO-Bind-Map. SNCA IRE hairpin RNA was 5′-end labeled with [γ-³²P]ATP and T4 kinase as previously described⁵. Approximately, 5 pmoles of ³²P labeled IRE RNA per reaction was folded in 1× RNase H buffer (New England Biolabs) by heating to 65° C. for 5 min and slowly cooling to room temperature. Serially diluted concentrations of Synucleozid were added to the RNA solutions and incubated at room temperature for 30 min. One of ASOs was then added to a final concentration of 500 nM, followed by incubation at room temperature for 30 min. Next, 0.05 U/μL RNase H (New England Biolabs) was added into each RNA-ligand mixture, and the samples were incubated for 20 min. Reactions were quenched by incubating at 65° C. for 20 min. T

Two ladders were generated to identify the positions of RNase H cleavage. Each guanosine (G) residue was identified by using RNase T1 under denaturing conditions as described above. Likewise, a hydrolysis ladder was generated as described above. Reactions were stopped by addition of an equal volume of 2× Loading Buffer. Fragments were separated on a denaturing 15% polyacrylamide gel and imaged using a BioRad PMI phosphorimager. Images were quantified with BioRad's QuantityOne software.

FRET-based ASO-Bind-Map. The SNCA IRE dually labeled with Cy3 and Cy5 dyes (100 nM) was folded by heating at 65° C. for 5 min in 1× Assay Buffer and slowly cooling to room temperature. BSA was added to the RNA solutions to a final concentration of 40 μg/mL followed by addition of compound. The mixtures were incubated at room temperature for 30 min. Fluorescence spectra were then acquired as a function of time (in kinetics mode) for 20 min using a Cary Eclips fluorescence spectrophotometer, with excitation and emission wavelengths of 532/10 nm and 668/5 nm, respectively. The ASO of interest was then added to a final concentration of 500 nM, and fluorescence spectra were acquired as described above.

Mapping Synucleozid's binding site in cells with ASO-Bind-Map. SH-SY5Y cells were treated with vehicle (DMSO) or Synucleozid at ˜60% confluency overnight and were then transfected with a gapmer ASO (200 nM) with Lipofectamine RNAiMAX (Invitrogen) according to the manufacturer's instructions. Mock samples were generated by treating cells with transfection reagent lacking oligonucleotide. RNA extraction, cDNA synthesis and RT-qPCR were performed as described above after 48 h. Primers for SNCA mRNA in this experiment cover sequences upstream and downstream of the IRE; forward primer: 5′-TTCAAGCCTTCTGCCTTTCCACCC-3′; reverse primer 5′-TCTCAGCAGCAGCCACAACT-CCCT-3′.

Polysome profiling. Polysome profiling studies were completed similarly to previously described methods (9, 10). SH-SY5Y cells were treated with DMSO vehicle or Synucleozid (1 μM) at 50% confluency. After 48 h, 100 μg/mL (final concentration) cycloheximide (CHX) was added to dishes, and cells were incubated at 37° C. for 10 min. After two times washing with ice-cold 1×DPBS supplemented with 100 μg/mL CHX, cells were scraped with ice-cold 300 μL 1×DPBS supplemented with 100 μg/mL CHX. Three 100 mm dishes of cells were prepared and combined as one profiling sample. After centrifugation, cell pellets were lysed with 250 μL of ice-cold polysome extraction buffer [10 mM NaCl, 10 mM MgCl₂, 10 mM Tris, pH 7.5, 1% Triton X-100, 1% sodium deoxycholate, 1 mM DTT supplemented with 0.1 mg/L CHX and 0.2 U/μL RNasin (Promega)]. The lysate was transferred to an ice-cold Eppendorf tube, gently vortexed and centrifuged for 15 min at 13,200 rpm, 4° C. The supernatant was layered onto 10 mL linear 10-50% sucrose gradients containing 20 mM HEPES (pH 7.4), 5 mM MgCl₂, 100 mM KCl, 300 mM DTf, and 100 g/mL CHX. The sucrose gradients were centrifuged at 40,000 rpm for 2 h at 4° C. then fractionated using a fraction collector (Brandel Inc.). The absorbance of cytosolic RNA was recorded at 254 nm by an inline UV monitor. A 400 μL aliquot from each fraction was collected. Then, RNA extraction, cDNA synthesis and RT-qPCR were performed as described above.

RNA immunoprecipitation. SH-SY5Y cells were cultured in 100 mm dishes to 60% confluency and treated with DMSO vehicle or Synucleozid (1 μM) for 48 h. Cells were washed with ice-cold 1×DPBS, harvested and lysed for 20 min on ice in 100 μL of M-PER mammalian protein extraction reagent supplemented with 1× Protease Inhibitor Cocktail III for Mammalian Cells (Research Products International Corp.) and 80 U RNaseOUT Recombinant Ribonuclease Inhibitor (Invitrogen) according to the manufacturer's instructions. The samples were centrifuged at 13.200 rpm for 15 min at 4° C. Supernatants were transferred into ice-cold Eppendorf tubes and incubated for overnight at 4° C. with Dynabeads Protein A (Life Technologies) that were pre-treated to bound to either O-actin mouse primary antibody (Cell Signaling: 8H10D10), IRP-1 or IRP-2 rabbit primary antibody (Cell Signaling: D6S4J and D6E6W). Beads were then washed three times with 1×DPBS with 0.02% Tween-20. Then, RNA extraction, cDNA synthesis and RT-qPCR were performed as described above. Relative RNA expression in IRP fraction and β-Actin fraction were determined by ΔΔCₜ method and normalized to 18S rRNA as an internal housekeeping gene. Normalized fold change was calculated by dividing relative SNCA mRNA expression in the cDNA library prepared from RNA extracted from the IRP immunoprecipitated fraction by the relative SNCA mRNA expression in the cDNA library prepared from RNA extracted from the β-actin immunoprecipitated fraction as shown in Eq. (3):

$$Normalized Fold Change = \frac{Relative RNA Expression in IRP fraction}{Relative RNA Expression in β - actin fraction} \qquad Eq . ( 3 )$$

In vitro displacement of IRPs by gel mobility shift assays. SNCA IRE hairpin RNA was 5′-end labeled with [γ-³²P]ATP as previously described⁵. Approximately, 2 pmoles of ³²P labeled RNA per 20 μL reaction was folded in 1× Assay Buffer by heating at 65° C. for 5 min and slowly cooling to room temperature. As indicated, vehicle, Synucleozid (0.2, 1, 5 μM) or unlabeled IRE RNA (1 μM, folded as described above and used as a positive control) was added to the samples, and the samples were incubated at room temperature for varying amounts of time (1-60 min as indicated). Then, 0.5 μg IRP-1 or IRP-2 (Abnova) was added, and the samples were incubated at room temperature for an additional 30 min. An equal volume of 2× Native Gel Loading Buffer (2×TBE, 50% Glycerol, 0.025% (w/v) Bromophenol blue and Xylene cyanol FF) was added to each sample. Note, Native Gel Loading Buffer added to Synucleozid-treated samples was supplemented with the same concentration of Synucleozid as in the sample to maintain a constant concentration. IRP:IRE complexes and free IRE were separated on a native 15% polyacrylamide prepared 1×TBE supplemented with the same concentration of Synucleozid as in compound-treated samples, and imaged using a Bio-Rad PMI phosphorimager. Images were quantified with BioRad's Quantity One software.

Treatment with Synucleozid and siRNA for proteomics profiling using Mass Spectrometry. SH-SY5Y cells were cultured in 100 mm dishes to 60% confluency and treated with 1.5 μM of Synucleozid or DMSO, or transfected with 0.1 μM α-synuclein siRNA (ON-TARGETplus Human SNCA siRNA-SMARTpool) or scramble siRNA (ON-TARGETplus Non-targeting siRNA) using Lipofectamine RNAiMAX (Invitrogen) according to the manufacturer's instructions for 48 h. Cells were gently washed twice with ice-cold PBS, detached and lysed in PBS via sonication. Western blot was performed as previously described to confirm α-synuclein protein was downregulated by Synucleozid and siRNA in these lysate samples (Fig. S13). Same samples were then denatured in 6 M urea in 50 mM NH₄HCO₃, reduced with 10 mM tris(2-carboxyethyl)phosphine hydrochloride (TCEP) for 30 min, and alkylated with 25 mM iodoacetamide for 30 min, in the dark at room temperature. Samples were diluted to 2 M urea with 50 mM NH₄HCO₃, and digested with trypsin (Thermo Scientific, 1.5 μL of 0.5 μg/μL) in the presence of 1 mM CaCl₂ (100× stock in water). The digestion was performed for 12 h at 37° C. Samples were acidified to a final concentration of 5% acetic acid, desalted over a self-packed C18 spin column, and dried. Samples were then analyzed by LC-MS/MS (see below). The MS data were processed with MaxQuant.

LC-MS/MS analysis. Peptides from samples described above were resuspended in water with 0.1% formic acid and analyzed using EASY-nLC 1200 nano-UHPLC coupled to Q Exactive HF-X Quadrupole-Orbitrap mass spectrometer (Thermo Scientific). The chromatography column consists 30 cm long, 75 μm i.d. microcapillary capped by a 5 μm tip and packed with ReproSil-Pur 120 C18-AQ 2.4 μm beads (Dr. Maisch GmbH). LC solvents had two buffers which were Buffer A (0.1% formic acid in H₂O) and Buffer B (0.1% formic acid in 90% MeCN:10% H₂O). Peptides from samples described above were eluted into the mass spectrometer at a flow rate of 300 nL/min over a 240 min linear gradient (5-35% Buffer B) at 65 C. Data-dependent mode (top-20, NCE 28, R=7500) was used to acquire data after full MS scan (R=60000, m/z 400-1300). Dynamic exclusion was 10 s. Peptide match was set to prefer. Isotope exclusion was enabled.

MaxQuant analysis. MaxQuant (11) (V1.6.1.0) was used to analyze the mass spectrometer data. Data was searched against the human proteome (Uniprot) and a list of contaminants (included in MaxQuant). The first and main peptide search tolerance were set to 20 ppm and 10 ppm respectively. Fragment mass tolerance was set to 0.02 Da. The false discovery rate (FDR) was 1% for proteins, peptides, and sites identification. The minimum peptide length was set to 6 amino acids (AA). Peptide requantification, label-free quantification (MaxLFQ), “match between runs” were all enabled. The minimal number of peptides for every protein was set to 2. A variable modification was set to be methionine oxidation, and a fixed modification was set to be carbamidomethylation of cysteines in searches.

RNA-Seq. SH-SY5Y cells were treated or transfected as described above for 48 h with Synucleozid (1.5 μM) and siRNA (0.1 μM). Total RNA was extracted using a miRNeasy Mini Kit (Qiagen, as described above) with on-column DNase I treatment. Qubit 2.0 Fluorometer (Invitrogen) was used for total RNA quantification. Quality of total RNA was assessed by using an Agilent Technologies 2100 Bioanalyzer RNA nano chip. Samples which have RNA integrity number >8.0 were used in future experiments. Probes provided by the NEBNext rRNA depletion module (catalog no.: E6310L, New England Biosciences) were used to deplete rRNA on approximate 500 ng of total RNA according to the manufacturer's recommendations. Library preparation was done with NEBNext Ultra 11 Directional RNA kit (catalog no.: E7760, New England Biosciences) according to the manufacturer's instructions. RNA samples were heated to 94° C. and simultaneously chemically fragmented in a divalent cation buffer for 15 min. Conversion of fragmented RNA to the first strand of cDNA was done with random hexamer priming and reverse transcription. To synthesize second strand, RNA template was removed and dUTP in place of dTTP was incorporated. The second strand was then end repaired and adenylated at the 3′ end. Ligation to the double-stranded cDNA was performed with a corresponding T nucleotide on the hairpin loop adaptor. dUTP in the loop as well as other incorporated U's in the second strand was removed by uracil-specific excision reagent (USER) enzyme. The directional sequencing of the original RNA was preserved by degradation of the second strand. Final libraries were generated from PCR amplification of the adaptor ligated DNA with Illumina barcoding primers. Only library fragments with both 5′ and 3′ adaptors were enriched in the final PCR amplification step. Final libraries were validated on a Bioanalyzer DNA chip. After validation, the final libraries were prepared to 2 nM, pooled equally, and finally sequenced on a Nextseq 500 v2.5 flow cell at 1.8 μM final concentration using 2×40 bp paired-end chemistry. Every sample with a base quality score >Q30 (<1 error per 1000 bp) generated around 20-25 million reads. Transcript abundance from these RNA samples were quantified using Kallisto (12). Gene level RNA-Seq differential expression analysis was done using the Sleuth package in R (13).

ScanFold analyses of mRNAs encoding intrinsically disordered proteins. The longest mRNA isoform of all human genes encoding intrinsically disordered proteins (IDPs) archived in the DisProt database were acquired from Ensembl BioMart (14). Each sequence was analyzed via ScanFold-Scan using a sliding window of 120 nt, a step size of 1 nt and 50 randomizations (used to calculate the thermodynamic z-score) (15). Predicted secondary structure models and z-scores were analyzed using ScanFold-Fold to weight all nt (and their structural contexts) by their contributions to low z-score windows (indicating sequences that are ordered to fold into unusually stable RNA structures). Calculated metrics are tabulated in Table S4.

### Experimental Descriptions Associated with Figures

FIGS. 1A-1D provide design and characterization of an SNCA 5′ UTR IRE targeting small molecule. FIG. 1A: Schematic depiction of α-synuclein-mediated disease pathway. FIG. 1B: Secondary structure of 5′ UTR IRE of SNCA mRNA that regulates translation, and the chemical structures of hit small molecules predicted by Inforna. FIG. 1C: Quantification of Western blotting screen of candidate small molecules inhibiting α-synuclein protein expression in SH-SY5Y neuroblastoma cells. FIG. 1D: Cytoprotective effect of Synucleozid against α-synuclein toxicity in SH-SY5Y cells measured using the Lactate Dehydrogenase (LDH) assay. Synucleozid abrogates cytotoxicity induced by α-synuclein pre-formed fibrils (PFFs), which act as seeds and recruit endogenous α-synuclein to aggregate. *, p<0.05: **, p<0.01; ***, p<0.001, as determined by ANOVA.

FIGS. 2A and 2B show effect Synucleozid on-target effects in cells. FIG. 2A Top: structures of designed luciferase (Luc) reporter plasmids used in selectivity studies. FIG. 2A Bottom: luciferase assay of Synucleozid effects in SH-SY5Y cells stably transduced with plasmids containing 5′ UTR of SNCA, amyloid precursor protein (APP), prion protein (PrP) or ferritin mRNAs. FIG. 2B: Selectivity of Synucleozid for inhibiting α-synuclein protein translation as compared to its effect on APP, PrP, ferritin, and transferrin receptor (TfR) determined by Western blotting. *, p<0.05; **, p<0.01; ***, p<0.001, as determined by ANOVA.

FIGS. 3A-3C show selectivity of Synucleozid for the A bulge in the SNCA IRE. FIG. 3A: Secondary structure of 2-AP labeled RNA used in the assays. FIG. 3B: Plot of the change in 2-AP fluorescence as a function of Synucleozid concentration. FIG. 3C: Plot of the affinity of Synucleozid for various SNCA IRE mutants as determined by competitive binding assays with the 2-AP-labeled RNA. RNA-0 is native SNCA IRE. RNA-12 is a fully base paired RNA in which all five internal bulges and loops have been mutated. Each of RNA-1 to RNA-5 has one bulge or loop mutated. RNA-6 to RNA-12 are mutants of the A bulge or have mutated closing base pairs. (Figs. S7 and S8).

FIGS. 4A-4C show that antisense oligonucleotide (ASO)-Bind-Map studies confirm that Synucleozid binds to the predicted site in vitro and in cells. FIG. 4A: Designed ASOs that tile through the SNCA IRE. FIG. 4B Left: scheme of RNase H mediated ASO-Bind-Map. FIG. 4B Right: relative cleavage of full length SNCA IRE by RNase H after hybridization of ASOs with or without Synucleozid pre-incubation. Statistical significance was calculated between each specific ASO with or without Synucleozid pre-incubation. FIG. 4C Left: scheme of FRET-based ASO-Bind-Map. FIG. 4C Right: normalized relative fold change of Cy5/Cy3 fluorescence for various ASOs with or without Synucleozid pre-incubation. **, p<0.01: ***, p<0.001, as determined by a two-tailed Student t-test.

FIGS. 5A, 5B show that cellular ASO-Bind-Map validates the SNCA IRE as the target of Synucleozid. FIG. 5A: Scheme of ASO-Bind-Map studies completed in cells. FIG. 5B: Expression of SNCA mRNA in SH-SY5Y cells transfected with designed gapmers. Synucleozid protects SNCA mRNA from RNase-mediated cleavage by ASO(1-10), which hybridizes with the Synucleozid binding site. Protection is not observed from cleavage mediated by ASO(29-39), which hybridizes to a distal site. *, p<0.05; **, p<0.01; ***, p<0.001, as determined by ANOVA.

FIGS. 6A-6C show an investigation of first potential mode of action of Synucleozid. FIG. 6A: Synucleozid could affect the loading of SNCA mRNA into polysomes and/or the assembly of active ribosomes, which can be studied by polysome profiling. Hypothesis: Synucleozid decreases density of ribosomes onto SNCA mRNA by stabilizing IRE and thus triggering steric hindrance with 40S pre-initiation complex. Experiments and Observations Study and Observations—Study by polysome profiling: 1. Synucleozid increases amount of α-synuclein mRNA bound to 40S: 2. Synycleozid decreases density of ribosomes bound to α-synuclein mRNA. FIG. 6B: Representative absorption trace (at 254 nm) of polysome fractionation from polysome profiling of SH-SY5Y cells treated with Synucleozid (1 μM) or vehicle (DMSO) (top) and quantification of the percentage of SNCA mRNA level in each fraction relative to total SNCA mRNA expression, as assessed by RT-qPCR (bottom). FIG. 6C: Percentage of SNCA mRNA present within monosome and polysome-containing fractions with (black) and without (white) Synucleozid (1 μM) treatment. Fractions labeled as “Incomplete Ribosome” contain 40S and 60S ribosomal subunits (fractions 1-5); “Single Ribosome” (fractions 6 and 7) indicates 80S ribosomes. *, p<0.05; **, p<0.01, as determined by a two-tailed Student t-test.

FIGS. 7A, 7B show investigation of second and third potential modes of action of Synucleozid. FIG. 7A: Synucleozid could affect the abundance of IRPs and/or the affinity of the IRP-IRE complex, which can be assessed by Western blotting and immunoprecipitation (IP). FIG. 7B: SNCA mRNA was pulled down from treated (Synucleozid; 1 μM) and untreated (Vehicle; DMSO) SH-SY5Y cells by immunoprecipitation of IRP-1 (black bars) or IRP-2 (white bars). SNCA mRNA levels were quantified by RT-qPCR. Iron (II) and Deferoxamine (DFOA) are positive controls for IRP-1, and ASO is positive control for IRP-2, to detect changes in the amount of immunoprecipitated mRNAs. The amount of SNCA mRNA bound to IRP-1 or IRP-2 show no significant difference with or without Synucleozid treatment. *, p<0.05; ***, p <0.001, as determined by a two-tailed Student t-test. Hypothesis:1. Synucleozid increases IRP-1 or 2 abundance; Synucleozid stabilized IRP-1 or 2 and SNCA mRNA complex. Experiments and Observations: Study by protein expression and IRP-1 or 2 immunoprecipitation; 1. Synucleozid does not affect IRP-1 or 2 abundance; 2. Immunoprecipitation of SNCA mRNAs bound to IRP-1 or 2 and RT-pPCT analysis shows Synucleozid does not affect IRP-1 or 2 recognition of SNCA mRNA.

FIGS. 8A-8C show global proteome profiles of SH-SY5Y cells after treatment with Synucleozid or an siRNA directed at α-synuclein. Volcano plots of SH-SY5Y cells treated with α-synuclein siRNA vs a scrambled control (0.1 μM), (FIG. 8A) or Synucleozid (1.5 μM) vs. vehicle (FIG. 8B) are shown. Data are represented as log 2 fold change: dotted lines represent a false discovery rate of 1% and an S0 of 0.1, indicating an adjusted p-value of 0.01. Red dots represent the common up-regulated proteins in the oxidative phosphorylation pathway. FIG. 8C: Venn diagrams showing down- or up-regulated proteins upon α-synuclein siRNA or Synucleozid treatment compared with their respective controls.

Figure S2 shows protein modulation with Synucleozid treatment. Western blot analysis for α-synuclein and other proteins that have IREs in their mRNA's UTR including APP, PrP, Ferritin and TfR with Synucleozid treatment. All panels were completed in SH-SY5Y cells, except for PrP protein which was assessed in Neuro-2A cells.

Figure S3 shows synucleozid effect on SNCA transcription. Synucleozid has no effect on SNCA mRNA expression in SH-SY5Y cells determined by RT-qPCR.

Figure S4 presents an In vitro translation assay showing the effect of Synucleozid against 5′ UTR of SNCA mRNA. The assay demonstrated that Synucleozid inhibited luciferase expression in a concentration-dependent manner when cells were transfected with SNCA-5′-UTR-Luc plasmids but not control plasmid lacking 5′ UTR. *, p<0.05; **, p<0.01: ***, p<0.001, as determined by ANOVA.

Figures S5A and S5B show cell viability with compound treatment. Synucleozid has no effect on (A) cell viability measured by MTS assay or (B) LDH release.

Figure S6 shows structures of IREs in mRNAs. Secondary structure of IREs in mRNAs of human SNCA, APP, PrP and H-Ferntin.

Figures S7A-S7C show competitive binding assay using 2-AP emission. Binding curves are shown: Fig S7A-RNA-0, RNA-12 and tRNA, FIG. 7B: RNA-1, -2, -3, 4, -5 and FIG. 7C: RNA-6, -7, -8, -9, -10, -11. The curves were obtained from competitive binding assay. See Fig. S8 for secondary structures of RNA-0 to RNA-12.

Figure S8 shows secondary structures of RNA competitors used in competitive binding assay. RNA-0 is native SNCA IRE RNA hairpin. Mutations of native IRE in RNA-1 to RNA-12 are shown in gray boxes.

Figure S9 shows thermal melting experiments with Synucleozid. Synucleozid only stabilized the wild type IRE upon binding by decreasing its ΔG_{37°} (from −2.91 to −3.23 kcal/mol) and increasing its Tₘ(from 51.4 to 54.8° C.) with no significant effect observed with the A-bulge-mutated RNA, showing that Synucleozid requires the display of the wild type A bulge in IRE RNA to affect its target.

Figures S10A-S10C show activity of Synucleozid derivatives (see also Table S1). Fig S10A: Chemical Structures of Synucleozid derivatives with CNS MPO scores in parentheses. SynucleoziD-NC is a negative control. Fig S10B: Activity of Synucleozid derivatives in 2-AP emission assay. Fig S10C: Activity of Synucleozid derivatives (5 μM) for inhibiting α-synuclein expression in SH-SY5Y cells assessed by Western blot. Note, Synucleozid was tested at 1 μM concentration. *, p<0.05; **, p<0.01 as determined by ANOVA.

Figures S11A and S11B show that RNase H-mediated ASO-Bind-Map confirms that Synucleozid binds to the predicted site in vitro. Fig S11A, Top: designed red ASOs hybridizing with A bulge nearby sequence within IRE RNA (RNase H cleavage sites are colored). Fig S11A Bottom: PAGE shows that Synucleozid binds to predicted site as demonstrated by protection of ³²P labeled IRE from cleavage when red ASOs hybridized with A bulge. Fig S11B, Top: designed blue ASOs hybridizing with sites other than A bulge within IRE RNA (RNase H cleavage sites are in colored boxes). Fig S11B, Bottom: PAGE shows that Synucleozid has no protection from cleavage when blue ASOs hybridized with sites other than A bulge at 1 μM. Lane T1 indicates cleavage by T1 nuclease under denaturing conditions (cleaves Gs). Lane OH indicates a hydrolysis ladder.

Figure S12 shows FRET-based ASO-Bind-Map with Synucleozid. Representative Cy5 emission time-dependent decay curves after adding ASOs to dual-labeled IRE hairpin RNA pre-incubated with or without Synucleozid (1 μM).

Figure S13 shows that Synucleozid has no effect on expression of IRP-1 or IRP-2. Western blot analysis was run to measure IRP-1 and IRP-2 expression with Synucleozid as well as siRNAs treatment. There were no changes of IRP-1 and IRP-2 expression in all conditions.

Figure S14 shows In vitro displacement of IRPs by gel mobility shift assays. Fig S14 Left and top right shows Representative gel images of synucleozid's effect on the binding of IRP-1 and IRP-2 to the SNCA IRE RNA. The amount of Synucleozid was varied from 0.2-5 μM, and its effect on IRP binding was studied over time (1-60 min). Unlabeled IRE RNA was used as a positive control to compete off ³²P-labeled IRE RNA binding to IRPs. Fig S14 graphs at Bottom right show quantification of synucleozid's effect on the binding of IRP-1 and IRP-2 to the SNCA IRE RNA from gel mobility shift assays. Neither Synucleozid concentration (0.2, 1, 5 μM) nor pre-incubation time (1, 5, 15, 60 min) has any significant effect on IRP binding/recognition.

Figure S15 shows Western blot of α-synuclein in proteomics samples with Synucleozid and siRNAs treatment. Western blot was run in SH-SY5Y cells with 48 h treatment of Synucleozid (1.5 IM), α-synuclein siRNA and Scramble siRNAs (0.1 μM). α-synuclein siRNA had around 70% inhibition while Synucleozid had 60% of α-synuclein. There was no inhibition in Scramble-siRNA-treated samples.

Figures S16A and S16B show Differential gene expression analysis in SH-SY5Y cells with Synucleozid and siRNAs treatment after 48 h. Volcano plots of all genes in the transcriptome of SH-SY5Y cells treated with α-synuclein siRNA vs a scrambled control (0.1 μM) as shown by Fig S16A, or by Fig S16B: Synucleozid (1.5 μM) vs vehicle (DMSO). Beta value is analogous to log 2 fold change. 19279 out of 19329 genes in Fig. A and 19979 out of 20034 genes in Fig. B were not significantly affected (adjusted p-value <0.05), demonstrating that Synucleozid and the α-synuclein siRNA have limited off-target effects. Red dot represents SNCA expression levels.

### TABLES

TABLE S1
Information of detectable transcripts^{a} in SH-SY5Y cells by RNA-Seq.

| | | | | | | Normalized |
|---|---|---|---|---|---|---|
| | | | | Contain | | Expression |
| Transcript | ENSTID | | Coded | the IRE | | (%) based |
| ID | (GRCh38) | Length | Protein | (Yes/No) | TPM ± SD | on TPM |
| SNCA-201 | ENST00000336904.7 | 3041 | 140aa | No | 0.11 ± 0.11 | 2.2 |
| SNCA-205 | ENST00000394991.7 | 1290 | 140aa | Yes | 1.76 ± 0.11 | 34.4 |
| SNCA-211 | ENST00000508895.5 | 905 | 140aa | No | 2.08 ± 0.02 | 40.7 |
| SNCA-207 | ENST00000502987.5 | 731 | 115aa | No | 0.47 ± 0.04 | 9.1 |
| SNCA-204 | ENST00000394989.6 | 3167 | 126aa | Yes | 0.69 ± 0.09 | 13.5 |

^{a}SNCA mRNAhas 13 transcripts in total. In the RNA-Seq experiment, 5 transcripts passed the defaulted detection filter (at least 5 estimated counts in at least 47% of the samples) (13).

TABLE S2
EC₅₀ values for Synucleozid and derivatives in a 2-AP binding assay
and percentage inhibition of α-synuclein expression in cells.

| | EC₅₀ in 2-AP | Percentage Inhibition of |
|---|---|---|
| Compound | assay/μM | α-synuclein expression |
| Synucleozid A | 2.72 ± 0.37 | 67.1 ± 3.8% (1 μM) |
| SynucleoziD-2 | 2.15 ± 0.53 | 38.0 ± 3.6% (5 μM) |
| SynucleoziD-3 | 2.54 ± 0.33 | 42.0 ± 7.2% (5 μM) |
| SynucleoziD-4 | 2.57 + 0.48 | 42.7 ± 8.3% (5 μM) |
| SynucleoziD-5 | 4.67 ± 0.35 | 29.7 ± 6.8% (5 μM) |
| SynucleoziD-NC | >20 | No Inhibition |

TABLE S3
Sequences of designed ASOs used in ASO-Bind-Map

| | ASO sequences | Gapmer ASO sequences |
|---|---|---|
| Compound | used in vitro^{a} | used in cells^{a,b} |
| ASO (1-10) | 5′-CACTCCCAGT-3′ | 5′-CACTCCCAGT-3′ |
| ASO (8-17) | 5′-GAATGGCCAC-3′ | |
| ASO (15-24) | 5′-TGTCGTCGAA-3′ | |
| ASO (22-31) | 5′-ACCACACTGT-3′ | |
| ASO (29-39) | 5′-TCCTTTACACC-3′ | 5′-TCCTTTACACC-3 |
| ASO (40-50) | 5′-GGCTAATGAAT-3′ | |
| ASO Control | | 5′-GUGAGGGUCA-3′ |

^{a}All nucleotides have phosphorothioate linkages.
^{b}2′-O-Methoxyelthyl (MOE) nucleotides are underlined.

TABLE S4
Summary of ScanFold results for mRNAs encoding intrinsically
disordered proteins. The SNCA mRNA is highlighted.

| | | | Delta | | % nt | %nt |
|---|---|---|---|---|---|---|
| Gene | nt | GC % | G | z-score | <−1 | <−2 |
| RPLP2 | 646 | 55.4% | −40.8 | −1.58 | 66.6% | 44.1% |
| IVL | 2152 | 58.7% | −36.3 | −1.46 | 75.7% | 22.7% |
| TCAP | 2123 | 62.0% | −42.7 | −1.29 | 79.2% | 17.4% |
| CD247 | 2922 | 57.1% | −36.2. | −1.28 | 61.6% | 29.9% |
| IKZF4 | 5313 | 50.9% | −30.1 | −1.27 | 66.1% | 27.0% |
| CHCHD5 | 3272 | 52.9% | −30.8 | −1.20 | 63.7% | 22.4% |
| PPP1R1 | 1731 | 51.9% | −30.1 | −1.16 | 57.9% | 30.1% |
| HMGA1 | 2159 | 61.3% | −38.5 | −1.15 | 71.2% | 21.5% |
| NUPR1 | 5491 | 50.3% | −30.5 | −1.14 | 62.3% | 31.4% |
| PRB4 | 916 | 56.6% | −26.2 | −1.07 | 49.8% | 22.6% |
| TRAPPC4 | 1924 | 46.1% | −29.8 | −1.02 | 67.7% | 16.0% |
| POU2AF1 | 2875 | 53.4% | −28.8 | −1.01 | 48.2% | 28.8% |
| DDIT3 | 1067 | 49.7% | −29.3 | −1.00 | 51.9% | 12.7% |
| CCL26 | 703 | 52.1% | −34.1 | −0.99 | 71.3% | 3.3% |
| PTP4A3 | 2785 | 62.6% | −44.8 | −0.95 | 60.9% | 17.3% |
| PNPO | 3657 | 51.4% | −32.8 | −0.93 | 52.9% | 20.5% |
| FCAR | 2476 | 45.7% | −27.9 | −0.91 | 52.8% | 16.6% |
| CCL21 | 864 | 58.1% | −35.7 | −0.91 | 59.4% | 16.6% |
| FIS1 | 1005 | 57.4% | −38.4 | −0.91 | 48.0% | 19.4% |
| STAT2 | 4985 | 50.5% | −31.7 | −0.91 | 55.0% | 14.2% |
| NKX3-1 | 3278 | 47.1% | −29.2 | −0.90 | 55.6% | 17.7% |
| SHC1 | 3481 | 56.5% | −37.3 | −0.89 | 50.9% | 17.8% |
| CAMP | 745 | 55.6% | −38.1 | −0.88 | 55.2% | 14.5% |
| CD4 | 3049 | 54.5% | −33.1 | −0.87 | 53.1% | 15.8% |
| PPP5C | 4675 | 55.2% | −35.7 | −0.87 | 52.6% | 16.4% |
| RELA | 3183 | 57.1% | −35.7 | −0.86 | 50.1% | 19.5% |
| GGA1 | 3205 | 62.0% | −40.6 | −0.83 | 48.0% | 15.9% |
| VAMP2 | 2126 | 53.2% | −28.8 | −0.83 | 54.5% | 17.4% |
| NABP2 | 1775 | 55.1% | −33.9 | −0.83 | 61.5% | 11.1% |
| DAXX | 2615 | 54.8% | −32.7 | −0.82 | 48.5% | 6.3% |
| DFFA | 6035 | 48.1% | −29.7 | −0.82 | 52.7% | 14.3% |
| VDR | 4773 | 54.2% | −33.1 | −0.81 | 55.0% | 15.0% |
| AKT2 | 5250 | 58.0% | −39.0 | −0.80 | 49.0% | 21.2% |
| ACPP | 3174 | 43.1% | −26.5 | −0.80 | 44.8% | 17.2% |
| CDKN1A | 2267 | 58.1% | −38.4 | −0.79 | 54.5% | 11.7% |
| HSDI7B1 | 4982 | 55.2% | −34.8 | −0.79 | 51.9% | 13.1% |
| NLRP1 | 5610 | 55.0% | −35.1 | −0.78 | 55.5% | 17.9% |
| VHL | 3737 | 47.3% | −30.7 | −0.77 | 47.0% | 18.2% |
| ADD1 | 5179 | 46.4% | −29.4 | −0.77 | 55.8% | 9.4% |
| SOST | 2296 | 54.3% | −35.8 | −0.76 | 47.9% | 10.0% |
| TP53 | 2724 | 52.5% | −31.6 | −0.75 | 52.2% | 5.7% |
| EMILIN1 | 3943 | 67.6% | −49.1 | −0.74 | 45.7% | 10.7% |
| CBY1 | 1269 | 50.9% | −31.7 | −0.74 | 59.7% | 6.6% |
| HYPK | 4777 | 50.1% | −29.4 | −0.73 | 46.0% | 6.1% |
| PAX5 | 8704 | 53.6% | −33.1 | −0.73 | 49.1% | 10.4% |
| AXIN1 | 6340 | 63.8% | −45.2 | −0.73 | 55.0% | 11.3% |
| NLGN3 | 3913 | 55.8% | −33.5 | −0.72 | 42.7% | 9.4% |
| EIF4EBP1 | 827 | 62.9% | −39.0 | −0.72 | 50.8% | 0.0% |
| MAP2K7 | 3430 | 62.9% | −40.7 | −0.72 | 40.6% | 14.5% |
| CSTB | 3744 | 53.5% | −35.7 | −0.71 | 50.3% | 6.1% |
| SNCG | 794 | 60.6% | −34.2 | −0.71 | 42.9% | 0.1% |
| DNAJC24 | 2970 | 37.8% | −22.9 | −0.71 | 47.6% | 16.3% |
| C1R | 3501 | 55.3% | −33.8 | −0.70 | 50.4% | 6.2% |
| SRP19 | 7063 | 43.2% | −24.2 | −0.70 | 43.8% | 11.7% |
| CACNAIS | 6028 | 56.0% | −34.9 | −0.68 | 48.9% | 15.9% |
| EPB41 | 6388 | 40.6% | −23.1 | −0.68 | 45.8% | 9.4% |
| FUS | 5119 | 49.8% | −31.9 | −0.68 | 40.2% | 7.8% |
| ABO | 6359 | 51.1% | −29.7 | −0.68 | 52.0% | 9.5% |
| ETF1 | 3874 | 43.5% | −27.3 | −0.68 | 40.9% | 10.0% |
| CDSN | 2555 | 56.5% | −31.5 | −0.67 | 41.5% | 0.0% |
| SP1 | 7680 | 44.9% | −25.7 | −0.67 | 44.5% | 6.4% |
| ADD2 | 9290 | 49.3% | −29.9 | −0.66 | 39.6% | 9.8% |
| PHYH | 1829 | 45.0% | −27.4 | −0.66 | 32.9% | 9.2% |
| SEM1 | 2773 | 31.8% | −19.9 | −0.65 | 47.1% | 5.0% |
| BCL2L1 | 2578 | 55.2% | −34.5 | −0.65 | 44.6% | 25.1% |
| EP300 | 8779 | 49.8% | −27.3 | −0.65 | 36.3% | 4.6% |
| ACTR8 | 3579 | 43.8% | −27.0 | −0.65 | 45.0% | 9.0% |
| SULT2B1 | 1447 | 59.9% | −37.0 | −0.63 | 36.3% | 8.4% |
| CRY AB | 3414 | 46.7% | −26.4 | −0.63 | 46.2% | 8.3% |
| PIM1 | 2703 | 56.5% | −36.1 | −0.62 | 48.3% | 5.2% |
| RAD52 | 2826 | 48.4% | −30.0 | −0.62 | 46.9% | 3.9% |
| SRPRA | 2986 | 51.5% | −31.1 | −0.62 | 41.7% | 3.3% |
| MAX | 3155 | 51.3% | −31.2 | −0.61 | 44.0% | 7.9% |
| CD3E | 2643 | 48.5% | −24.7 | −0,61 | 39.0% | 1.9% |
| SERPINE1 | 3190 | 51.6% | −31.5 | −0.61 | 36.7% | 10.8% |
| UBE2Z | 4250 | 46.5% | −28.4 | −0.60 | 47.8% | 8.4% |
| SSB | 2463 | 40.0% | −23.2 | −0.60 | 40.6% | 12.0% |
| APEX1 | 1803 | 50.2% | −30.7 | −0.60 | 26.5% | 1.2% |
| WEE1 | 5187 | 37.9% | −22.3 | −0.60 | 36.1% | 8.5% |
| RAD23A | 1871 | 58.3% | −35.3 | −0.59 | 41.8% | 20.1% |
| PLK1 | 4135 | 57.1% | −36.4 | −0.59 | 48.3% | 7.1% |
| CALR | 1903 | 53.9% | −30.4 | −0.59 | 42.3% | 11.6% |
| GAP43 | 1901 | 47.6% | −24.3 | −0.59 | 41.6% | 4.6% |
| POU2F1 | 14332 | 41.8% | −24.2 | −0.59 | 41.4% | 8.1% |
| RAF1 | 3300 | 51.3% | −32.3 | −0.59 | 38.2% | 4.7% |
| MDM2 | 12632 | 41.9% | −25.2 | −0.58 | 44.8% | 8.0% |
| AR | 10676 | 48.0% | −28.4 | −0.58 | 41.2% | 7.9% |
| CD3G | 2690 | 40.2% | −22.4 | −0.58 | 36.6% | 9.5% |
| SULT1A3 | 1326 | 54.6% | −34.9 | −0.58 | 35.3% | 0.3% |
| CHCHD4 | 1603 | 48.8% | −30.0 | −0.58 | 42.0% | 3.1% |
| FNTA | 7327 | 38.9% | −22.9 | −0.57 | 38.8% | 7.8% |
| CRAT | 2768 | 62.1% | −39.6 | −0.57 | 40.3% | 13.0% |
| SNN | 3264 | 53.7% | −34.0 | −0.57 | 40.6% | 8.7% |
| PRLR | 11581 | 38.4% | −21.4 | −0.57 | 43.6% | 10.5% |
| KCNE1 | 3505 | 50.6% | −29.5 | −0.57 | 40.5% | 7.0% |
| CRY2 | 4204 | 57.0% | −36.7 | −0.57 | 30.6% | 8.0% |
| LTF | 2979 | 53.5% | −35.0 | −0.57 | 42.0% | 7.5% |
| NR112 | 4417 | 50.5% | −30.0 | −0.56 | 47.1% | 5.2% |
| XRCC4 | 1696 | 36.7% | −20.8 | −0.56 | 37.8% | 6.3% |
| CACYBP | 2835 | 39.5% | −22.3 | −0.55 | 42.5% | 10.5% |
| PPARG | 2029 | 44.0% | −25.8 | −0.55 | 31.5% | 3.1% |
| UBA2 | 4005 | 43.8% | −26.8 | −0.54 | 58.4% | 6.5% |
| AHR | 6958 | 38.2% | −21.8 | −0.54 | 33.7% | 6.6% |
| FXN | 6978 | 44.5% | −25.7 | −0.54 | 40.1% | 10.1% |
| GMNN | 1263 | 43.6% | −25.8 | −0.54 | 29.8% | 0.0% |
| GRB2 | 3729 | 52.3% | −34.1 | −0.53 | 39.9% | 8.0% |
| SMAD4 | 8769 | 41.1% | −25.4 | −0.53 | 37.6% | 9.5% |
| NKD2 | 2155 | 67.5% | −43.2 | −0.53 | 44.3% | 1.0% |
| TDG | 3183 | 39.0% | −23.7 | −0.53 | 39.8% | 2.0% |
| HMGN2 | 1940 | 44.1% | −26.4 | −0.53 | 36.6% | 12.2% |
| TNNI3 | 992 | 58.5% | −36.9 | −0.53 | 49.6% | 7.4% |
| SNW1 | 2163 | 44.9% | −25.0 | −0.53 | 34.0% | 5.1% |
| NR3C1 | 7286 | 39.1% | −22 9 | −0.52 | 39.1% | 2.3% |
| FGF12 | 5406 | 34.0% | −19.0 | −0.51 | 32.6% | 5.9% |
| PTHLH | 1891 | 47.6% | −25.9 | −0.51 | 40.9% | 0.0% |
| FHOD1 | 4321 | 59.8% | −38.1 | −0.51 | 39.9% | 8.4% |
| MBP | 9797 | 51.5% | −31.8 | −0.51 | 36.2% | 6.6% |
| BRCA1 | 7270 | 41.9% | −23.5 | −0.51 | 37.8% | 4.9% |
| TP73 | 5192 | 61.1% | −38.9 | −0.51 | 36.7% | 8.6% |
| GHR | 4899 | 42.6% | −25.0 | −0.50 | 33.3% | 2.5% |
| PRNP | 2657 | 47.1% | −29.7 | −0.50 | 41.0% | 15.9% |
| PTGES3 | 2939 | 42.9% | −27 2 | −0.50 | 34.1% | 5.3% |
| CRK | 3850 | 45.2% | −28.1 | −0.50 | 35.5% | 4.5% |
| RXRA | 5770 | 60.6% | −40.3 | −0.49 | 40.3% | 4.7% |
| EIF1AX | 4414 | 35.1% | −21.0 | −0.49 | 38.2% | 7.7% |
| EIF1 | 2338 | 46.4% | −27.0 | −0.49 | 42.8% | 5.9% |
| ATP7A | 8492 | 38.9% | −22.6 | −0.49 | 39.9% | 7.5% |
| LMNA | 3178 | 61.5% | −40.0 | −0.49 | 36.5% | 13.7% |
| MYC | 3721 | 48.8% | −27.8 | −0.49 | 33.2% | 9.5% |
| EWSR1 | 7285 | 42.4% | −25.4 | −0.48 | 35.1% | 7.5% |
| NCOA3 | 7961 | 42.7% | −24.5 | −0.48 | 42.5% | 4.1% |
| NPPB | 708 | 56.8% | −38.4 | −0.48 | 7.1% | 0.0% |
| KCNAB1 | 4437 | 42.9% | −26.1 | −0.48 | 32.5% | 1.8% |
| CCLII | 1079 | 45.0% | −24.5 | −0.48 | 34.2% | 21.8% |
| IGFBP6 | 1177 | 64.1% | −44.1 | −0.48 | 39.3% | 14.5% |
| NCBP1 | 4983 | 40.3% | −23.5 | −0.48 | 36.6% | 7.5% |
| PIP4K2B | 5734 | 53.3% | −33.3 | −0.47 | 41.8% | 6.3% |
| PSAP | 2830 | 54.4% | −36.0 | −0.46 | 44.8% | 3.2% |
| GSK3B | 7711 | 42.1% | −24.3 | −0.46 | 37.7% | 8.1% |
| PLG | 2741 | 49.5% | −29.1 | −0.46 | 32.6% | 0.8% |
| APC | 10704 | 39.6% | −21.0 | −0.46 | 34.9% | 7.3% |
| TMSB4X | 1702 | 52.8% | −33.9 | −0.46 | 35.3% | 1.8% |
| EZR | 3068 | 50.7% | −30.3 | −0.46 | 34.0% | 6.9% |
| MAPT | 6816 | 57.4% | −34.3 | −0.46 | 35.9% | 4.7% |
| MECP2 | 10467 | 52.1% | −31.1 | −0.45 | 37.1% | 6.7% |
| PAD14 | 2267 | 57.0% | −35.9 | −0.45 | 37.5% | 7.9% |
| NPPA | 855 | 55.1% | −35.6 | −0.45 | 34.0% | 5.3% |
| ATXN3 | 6950 | 37.9% | −22.7 | −0.45 | 35.3% | 8.6% |
| CGB3 | 877 | 65.6% | −40.6 | −0.45 | 65.9% | 3.4% |
| RYBP | 7215 | 40.6% | −23.6 | −0.45 | 39.3% | 3.1% |
| CAST | 4506 | 42.1% | −22.2 | −0.44 | 28.6% | 6.2% |
| BASP1 | 1807 | 54.3% | −30.2 | −0.44 | 32.5% | 10.2% |
| KITLG | 5737 | 37.3% | −22.8 | −0.44 | 29.8% | 4.6% |
| MLLT3 | 6724 | 38.0% | −21.3 | −0.44 | 36.4% | 7.6% |
| FOS | 2104 | 52.8% | −31.6 | −0.43 | 31.0% | 2.8% |
| ESR1 | 6466 | 46.1% | −27.7 | −0.43 | 32.0% | 4.9% |
| GATM | 3911 | 45.8% | −27.8 | −0.42 | 34.1% | 3.1% |
| EGFR | 9905 | 47.8% | −27.8 | −0.42 | 34.8% | 7.6% |
| CAD | 7286 | 57.7% | −37.6 | −0.42 | 36.4% | 5.6% |
| CD69 | 1676 | 36.5% | −21.6 | −0.41 | 30.0% | 4.2% |
| PPP1R2 | 3386 | 38.2% | −22.7 | −0.41 | 35.9% | 10.0% |
| WRN | 7353 | 38.7% | −21.7 | −0.40 | 33.3% | 3.3% |
| TCIM | 1854 | 36.2% | −20.7 | −0.40 | 22.9% | 0.0% |
| PEX5 | 3252 | 54.3% | −35.7 | −0.39 | 37.9% | 6.7% |
| MICA | 2260 | 55.0% | −36.0 | −0.39 | 35.1% | 3.6% |
| SNCA | 3167 | 38.2% | −21.8 | −0.37 | 36.0% | 4.6% |
| SECISBP2 | 5405 | 41.4% | −24.3 | −0.37 | 31.2% | 3.5% |
| ZFYVE9 | 5194 | 45.5% | −27.1 | −0.37 | 29.6% | 3.0% |
| PTN | 1614 | 43.2% | −22.9 | −0.37 | 31.0% | 8.2% |
| PPPIR8 | 2651 | 48.4% | −28.7 | −0.37 | 32.5% | 4.5% |
| BCL2 | 7461 | 45.6% | −27.7 | −0.36 | 29.4% | 5.2% |
| CFTR | 6132 | 41.0% | −23.6 | −0.36 | 28.6% | 5.3% |
| CTDP1 | 5866 | 61.3% | −41.0 | −0.36 | 31.0% | 2.6% |
| CCL1 | 592 | 49.5% | −28.6 | −0.36 | 5.4% | 0.0% |
| KDM5B | 10341 | 44.4% | −25.5 | −0.35 | 32.2% | 6.0% |
| CDKN1B | 2411 | 45.9% | −28.6 | −0.35 | 24.1% | 8.3% |
| ESR2 | 5458 | 48.6% | −30.1 | −0.35 | 37.7% | 9.3% |
| DDX4 | 2884 | 40.1% | −23.6 | −0.35 | 31.3% | 3.2% |
| CUTA | 1378 | 55.6% | −34.4 | −0.35 | 36.6% | 19.9% |
| LICAM | 5141 | 60.0% | −36.9 | −0.34 | 26.8% | 6.0% |
| TOP1 | 3734 | 42.1% | −21.9 | −0.34 | 31.6% | 2.8% |
| YAP1 | 5401 | 43.5% | −26.4 | −0.34 | 35.1% | 6.8% |
| UPF2 | 5569 | 40.3% | −21.0 | −0.33 | 34.9% | 3.6% |
| PCP4 | 706 | 41.9% | −20.0 | −0.30 | 26.6% | 0.0% |
| RPA1 | 4340 | 48.2% | −28.8 | −0.30 | 28.5% | 2.3% |
| RTN4 | 4697 | 43.9% | −24.4 | −0.29 | 31.8% | 7.0% |
| FMR1 | 4830 | 35.7% | −20.6 | −0.29 | 29.5% | 6.9% |
| PKIA | 3962 | 37.2% | −21.1 | −0.29 | 27.2% | 9.3% |
| STAT1 | 4310 | 43.6% | −25.4 | −0.29 | 27.2% | 2.1% |
| SAE1 | 2673 | 49.5% | −30.0 | −0.29 | 28.8% | 6.0% |
| NFKBIA | 1558 | 53.1% | −32.8 | −0.28 | 37.5% | 6.9% |
| HTRA2 | 2370 | 60.5% | −41.8 | −0.28 | 36.9% | 14.1% |
| RAP2A | 5540 | 38.4% | −23.5 | −0.28 | 26.8% | 3.2% |
| MBD2 | 5064 | 45.1% | −26.1 | −0.27 | 31.8% | 6.9% |
| FCERIG | 590 | 48.1% | −23.2 | −0.27 | 30.5% | 0.0% |
| PPP3CA | 4743 | 42.9% | −25.5 | −0.26 | 23.1% | 3.2% |
| TCF4 | 8343 | 38.2% | −21.3 | −0.25 | 30.8% | 4.8% |
| UROD | 2138 | 57.0% | −34.4 | −0.25 | 25.0% | 6.7% |
| NCBP2 | 4161 | 40.3% | −23.4 | −0.24 | 23.1% | 1.2% |
| AGO2 | 14595 | 46.6% | −28.1 | −0.23 | 23.5% | 5.4% |
| GRB14 | 2382 | 51.8% | −30.8 | −0.23 | 18.3% | 3.0% |
| PPP3R1 | 3024 | 40.7% | −22.5 | −0.23 | 31.0% | 5.9% |
| IBSP | 1573 | 42.5% | −20.6 | −0.22 | 34.1% | 5.9% |
| RALA | 2787 | 41.0% | −23.6 | −0.21 | 31.6% | 5.5% |
| TYMS | 1613 | 48.9% | −29.6 | −0.21 | 19.3% | 9.5% |
| CCNH | 2391 | 35.2% | −18.9 | −0.21 | 31.5% | 8.3% |
| MMP12 | 1874 | 39.1% | −21.8 | −0.20 | 28.5% | 3.3% |
| COL2A1 | 5059 | 62.3% | −42.1 | −0.19 | 32.8% | 3.8% |
| CD3D | 861 | 51.8% | −30.9 | −0.18 | 25.3% | 0.2% |
| SPP1 | 1664 | 41.8% | −22.1 | −0.18 | 33.3% | 7.0% |
| TOBI | 2238 | 42.2% | −23.0 | −0.17 | 19.0% | 1.3% |
| PITGI | 1070 | 49.3% | −2.8.2 | −0.17 | 40.4% | 4.5% |
| STMN1 | 2947 | 39.3% | −2.0.9 | −0.17 | 26.5% | 4.5% |
| XPA | 1584 | 42.4% | −23.3 | −0.16 | 21.7% | 0.9% |
| RHEB | 2046 | 48.0% | −31.4 | −0.15 | 18.3% | 1.6% |
| HNRNPA1 | 4121 | 43.0% | −24.1 | −0.15 | 26.1% | 3.6% |
| MYOM1 | 5847 | 49.3% | −28.7 | −0.14 | 22.3% | 2.9% |
| CCNB1 | 2029 | 41.9% | −23.4 | −0.14 | 21.5% | 2.0% |
| NOTCH1 | 9568 | 63.5% | −41.0 | −0.12 | 23.0% | 3.1% |
| UAP1 | 2377 | 44.8% | −26.9 | −0.11 | 16.7% | 5.0% |
| TCF7L2 | 4136 | 45.8% | −22.0 | −0.11 | 23.0% | 5.9% |
| HIF1A | 3956 | 36.4% | −18.7 | −0.06 | 22.7% | 2.6% |
| JAGI | 5940 | 49.6% | −30.0 | −0.06 | 24.0% | 3.2% |
| SPRR2E | 692 | 49.7% | −24.6 | −0.05 | 33.2% | 1.0% |
| INSM1 | 2846 | 63.2% | −43.1 | −0.05 | 24.6% | 0.4% |
| ADRM1 | 1530 | 64.2% | −41.5 | −0.01 | 13.8% | 0.0% |
| SOD1 | 1746 | 53.7% | −36.7 | −0.01 | 21.4% | 0.6% |
| CDKN1C | 2051 | 63.8% | −43.0 | −0.01 | 14.2% | 0.8% |
| CITED2 | 2382 | 48.1% | −27.7 | 0.00 | 17.9% | 4.3% |
| NEUROG1 | 1683 | 60.2% | −35.8 | 0.07 | 6.1% | 0.0% |
| USP7 | 5831 | 48.6% | −29.1 | 0.11 | 19.0% | 3.0% |
| SNCB | 1407 | 66.1% | −38.8 | 0.12 | 19.3% | 1.7% |
| GADD45A | 1352 | 50.7% | −31.3 | 0.18 | 10.6% | 3.3% |
| HRAS | 1233 | 65.8% | −41.9 | 0.23 | 13.8% | 6.7% |
| COX17 | 684 | 43.1% | −23.1 | 0.37 | 12.1% | 0.0% |
| ZNF593 | 698 | 62.0% | −38.5 | 0.37 | 14.0% | 0.9% |

TABLE SS
Primers used to generate plasmid constructs

| Primer # | Primer name | Sequence |
|---|---|---|
| 1 | pIRES-LUC F | 5′GGACTCAGATCTCGAGATGGAAGATGCCAAAAAC |
| 2 | pIRES-LUC R | 5′GAAGCTTGAGCTCGATTACACGGCGATCTTGCC |
| 3 | pIRES-SYN-LUC pF | 5′GGACTCAGATCTCGAGAGGAGAAGGAGAAGGAGG |
| 4 | pIRES-SYN-LUC pR | 5′CATCTTCCATCTCGAGCCATGGCTAATGAATTCC |
| 5 | pIRES-APP-LUC pF | 5′GGACTCAGATCTCGAGGGATCAGCTGACTCGCCT |
| 6 | pIRES-APP-LUC pR | 5′CATCTTCCATCTCGAGTGCCAAACCGGGCAGCAT |
| 7 | pIRES-SYN-LUC F | 5′AGGAATTCATTAGCCATGGAAGATGCCAAAAAC |
| 8 | pIRES-SYN-LUC R | 5′GGCTAATGAATTCCTTTACACCACACTGTCGTC |
| 9 | pIRES-APP-LUC F | 5′CGCGACCCTGCGCGGGGCACCGAGTGCGCTGCT |
| 10 | pIRES-APP-LUC R | 5′CCGCGCAGGGTCGCGATGGAAGATGCCAAAAAC |
| 11 | pCDH-SYN-LUC F | 5′CCACCGGTCGGGATCCAGGAGAAGGAGAAGGAGG |
| 12 | pCDH-SYN-LUC R | 5′GGATCAATTGCGGCCGCTTACACGGCGATCTTGCC |
| 13 | pCDH-APP-LUC F | 5′CGCGACCCTGCGCGGGGCACCGAGTGCGCTGCT |
| 14 | pCDH-APP-LUC R | 5′GGATCAATTGCGGCCGCTTACACGGCGATCTTGCC |
| 15 | pCDH-LUC F | 5′CCACCGGTCGGGATCCATGGAAGATGCCAAAAA |
| 16 | pCDH-LUC R | 5′GGATCAATTGCGGCCGCTTACACGGCGATCTTGCC |
| 17 | pCDH-PrP-LUC | 5′CCACCGGTCGGGATCCAGCTTCCCCCTCGGCCCC |
| | sense oligonucleotides | GCGCGTCGCCTGTCCTCCGAGCCAGTCGCTGACAG |
| | (119 bases) | CCGCGGCGCCGCGAGCTTCTCCTCTCCTCACGACC |
| | | GAGAGCAGTCATTAC |
| 18 | pCDH-PrP-LUC | 5′GTTTAAACGCTAGCGGATCCCATGGTAATGACT |
| | antisense | GCTCTCGGTCGTGAGGAGAGGAGAAGCTCGCGGC |
| | oligonucleotides (123 | GCCGCGGCTGTCAGCGACTGGCTCGGAGGACAGG |
| | bases) | CGACGCGCGGGGCCGAGGGGGA |
| 19 | pCDH-FRT-LUC F | 5′CCACCGGTCGGGATCCCAGACGTTCTTCGCCGA |
| | | GAGTCGT |
| 20 | pCDH-FRT-LUC R | 5′CATCTTCCATGGATCCGGCGGCGACTAAGGAGA |
| | | GGGCGGC |

### REFERENCES

1. Clamp, M.; Fry, B.; Kamal, M.; Xie, X.; Cuff, J.; Lin, M. F.: Kellis, M.; Lindblad-Toh, K.; Lander, E. S., Distinguishing protein-coding and noncoding genes in the human genome. Proc. Natl. Acad Sci. U.S.A. 2007, 104 (49), 19428-33.
2. Hopkins, A. L.; Groom, C. R., The druggable genome. Nat. Rev. Drug Discov. 2002, 1 (9), 727-30.
3. Dang, C. V.; Reddy, E. P.; Shokat, K. M.; Soucek, L., Drugging the ‘undruggable’ cancer targets. Nat. Rev. Cancer 2017, 17 (8), 502-508.
4. Spiegel, J.; Cromm, P. M.; Zimmermann, G.; Grossmann, T. N.; Waldmann, H., Small-molecule modulation of Ras signaling. Nat. Chem. Biol. 2014, 10 (8), 613-22.
5. Velagapudi, S. P.: Gallo, S. M.; Disney, M. D., Sequence-based design of bioactive small molecules that target precursor microRNAs. Nat. Chem. Biol. 2014, 10 (4), 291-7.
6. Velagapudi, S. P.; Cameron, M. D.: Haga, C. L.; Rosenberg, L. H.; Lafitte, M.; Duckett, D. R.; Phinney, D. G.; Disney, M. D., Design of a small molecule against an oncogenic noncoding RNA. Proc. Natl. Acad. Sci. U.S.A 2016, 113 (21), 5898-903.
7. Lee, V. M.; Trojanowski, J. Q., Mechanisms of Parkinson's disease linked to pathological alpha-synuclein: new targets for drug discovery. Neuron 2006, 52(1), 33-8.
8. Spillantini, M. G.: Schmidt, M. L.; Lee, V. M.; Trojanowski, J. Q.: Jakes, R.; Goedert, M., Alpha-synuclein in Lewy bodies. Nature 1997, 388 (6645), 83940.
9. Luk, K. C.; Kehm, V.; Carroll, J.; Zhang, B.; O'Brien, P.; Trojanowski, J. Q.; Lee, V. M., Pathological alpha-synuclein transmission initiates Parkinson-like neurodegeneration in nontransgenic mice. Science 2012, 338 (6109), 949-53.
10. Junn, E.: Mouradian, M. M., Human alpha-synuclein over-expression increases intracellular reactive oxygen species levels and susceptibility to dopamine. Neurosci. Lett. 2002, 320 (3), 146-50.
11. Rockenstein, E., Nuber, S.: Overk, C. R.; Ubhi, K.: Mante, M.: Patrick, C.: Adame, A.; Trejo-Morales, M.; Gerez, J.; Picotti, P.; Jensen, P. H.; Campioni, S.; Riek, R.; Winkler, J.; Gage, F. H.: Winner, B.; Masliah, E., Accumulation of oligomer-prone alpha-synuclein exacerbates synaptic and neuronal degeneration in vivo. Brain 2014, 137 (Pt 5), 1496-513.
12. Singleton. A. B.; Farrer. M.; Johnson, J.; Singleton, A.; Hague. S.; Kachergus, J.; Hulihan, M.: Peuralinna, T.; Dutra, A.; Nussbaum, R.; Lincoln, S.; Crawley, A.: Hanson, M., Maraganore, D.: Adler, C.: Cookson, M. R.; Muenter, M.; Baptista. M.; Miller, D.: Blancato, J.: Hardy, J.; Gwinn-Hardy, K., alpha-Synuclein locus triplication causes Parkinson's disease. Science 2003, 302 (5646), 841.
13. Maraganore, D. M.; de Andrade, M.; Elbaz, A.; Farrer, M. J.: Ioannidis, J. P.: Kruger, R.; Rocca, W. A.: Schneider, N. K.; Lesnick, T. G.; Lincoln, S. J.; Hulihan, M. M.; Aasly, J. O.; Ashizawa, T.; Chartier-Harlin, M. C.: Checkoway, H.; Ferrarese, C.: Hadjigeorgiou, G.: Hattori, N.; Kawakami, H.; Lambert, J. C.; Lynch. T.: Mellick, G. D.; Papapetropoulos, S.; Parsian, A.: Quattrone, A.; Riess, O., Tan, E. K.; Van Broeckhoven, C.; Genetic Epidemiology of Parkinson's Disease Consortium, Collaborative analysis of alpha-synuclein gene promoter variability and Parkinson disease. JAMA 2006, 296 (6), 661-70.
14. Fuchs, J.; Tichopad, A.; Golub, Y.; Munz, M.: Schweitzer. K. J.; Wolf, B.: Berg, D.; Mueller, J. C.; Gasser, T., Genetic variability in the SNCA gene influences alpha-synuclein levels in the blood and brain. FASEB J. 2008, 22 (5), 1327-34.
15. Soldner, F.; Stelzer, Y.: Shivalila, C. S.; Abraham, B. J.: Latourelle, J. C.; Barrasa, M. I.: Goldmann, J.: Myers, R. H.; Young, R. A.: Jacnisch, R., Parkinson-associated risk variant in distal enhancer of alpha-synuclein modulates target gene expression. Nature 2016, 533 (7601), 95-9.
16. Junn, E.: Lee, K. W.; Jeong, B. S.; Chan, T. W.; Im, J. Y.; Mouradian, M. M., Repression of alpha-synuclein expression and toxicity by microRNA-7. Proc. Natl. Acad. Sci. U.S.A. 2009, 106 (31), 13052-7.
17. Maraganore, D. M., Rationale for therapeutic silencing of alpha-synuclein in Parkinson's disease. J. Mov. Disord 2011, 4 (1), 1-7.
18. Friedlich, A. L.; Tanzi, R. E.; Rogers, J. T., The 5′-untranslated region of Parkinson's disease alpha-synuclein messengerRNA contains a predicted iron responsive element. Mol. Psychiatry 2007, 12 (3), 222-3.
19. Febbraro, F.; Giorgi, M.; Caldarola, S.; Loreni, F.; Romero-Ramos, M., alpha-Synuclein expression is modulated at the translational level by iron. Neuroreport 2012, 23 (9), 576-80.
20. McDowall, J. S.; Brown, D. R., Alpha-synuclein: relating metals to structure, function and inhibition. Metallomics 2016, 8 (4), 385-97.
21. Zhou, Z. D.: Tan, E. K., Iron regulatory protein (IRP)-iron responsive element (IRE) signaling pathway in human neurodegenerative diseases. Mol. Neurodegener. 2017, 12(1), 75.
22. Olivares, D.: Huang, X.; Branden, L.; Greig, N. H.; Rogers, J. T., Physiological and pathological role of alpha-synuclein in Parkinson's disease through iron mediated oxidative stress; the role of a putative iron-responsive element. Int. J. Mol. Sci. 2009, 10 (3), 1226-60.
23. Castellani, R. J.; Siedlak, S. L.; Perry, G.: Smith, M. A., Sequestration of iron by Lewy bodies in Parkinson's disease. Acta Neuropathol. 2000, 100 (2), 1114.
24. Disney, M. D.; Winkelsas, A. M.: Velagapudi, S. P.; Southern, M.; Fallahi, M.; Childs-Disney, J. L., Infoma 2.0: a platform for the sequence-based design of small molecules targeting structured RNAs. ACS Chem. Biol. 2016, 11 (6), 1720-8.
25. Rzuczek, S. G.: Colgan, L. A.; Nakai, Y.; Cameron, M. D.; Furling, D.: Yasuda, R.; Disney, M. D., Precise small-molecule recognition of a toxic CUG RNA repeat expansion. Nat. Chem. Biol. 2017, 13 (2), 188-193.
26. Costales, M. G.: Haga. C. L.: Velagapudi, S. P.; Childs-Disney, J. L.; Phinney, D. G.; Disney, M. D., Small molecule inhibition of microRNA-210 reprograms an oncogenic hypoxic circuit. J. Am. Chem. Soc. 2017, 139 (9), 3446-3455.
27. Childs-Disney, J. L.; Tran, T.; Vummidi, B. R.; Velagapudi, S. P.: Haniff, H. S.; Matsumoto, Y.; Crynen, G.; Southern, M. R.; Biswas, A.; Wang, Z.-F.; Tellinghuisen, T. L.: Disney, M. D., A massively parallel selection of small molecule-RNA motif binding partners informs design of an antiviral from sequence. Chem 2018, 4 (10), 2384-2404.
28. Gene SNCA. Bethesda (Md.): National Library of Medicine (US), National Center for Biotechnology Information; 2004-2019 Sep. 4. Available from: https://www.ncbi.nlm.nih.gov/gene/
29. Pimentel, H.: Bray, N. L.: Puente, S.: Meisted, P.; Pachter, L., Differential analysis of RNA-seq incorporating quantification uncertainty. Nat. Methods 2017, 14 (7), 687-690.
30. Rogers, J. T.: Mikkilineni, S.; Cantuti-Castelvetri, I.; Smith, D. H., Huang, X.; Bandyopadhyay, S.: Cahill, C. M.: Maccecchini, M. L.; Lahiri, D. K.; Greig, N. H., The alpha-synuclein 5′untranslated region targeted translation blockers: anti-alpha synuclein efficacy of cardiac glycosides and Posiphen. J. Neural Transm. 2011, 118 (3), 493-507.
31. Kalvari, I.; Argasinska, J.: Quinones-Olvera, N.; Nawrocki, E. P.: Rivas, E.; Eddy, S. R.; Bateman, A.: Finn, R. D.; Petrov, A. I., Rfam 13.0: shifting to a genome-centric resource for non-coding RNA families. Nucleic Acids Res. 2018, 46 (DI), D335-d342.
32. Gene SNCA. Bethesda (Md.): National Library of Medicine (US), National Center for Biotechnology Information; 2004-2019 Sep. 4. Available from: https://www.ncbi.nlm.nih.gov/gene/
33. Volpicelli-Daley, L. A.; Luk, K. C.; Patel, T. P.: Tanik, S. A.: Riddle, D. M.; Stieber, A.; Meaney, D. F.: Trojanowski, J. Q.; Lee, V. M., Exogenous alpha-synuclein fibrils induce Lewy body pathology leading to synaptic dysfunction and neuron death. Neuron 2011, 72 (1), 57-71.
34. Rupani, D. N.; Connell, G. J., Transferrin receptor mRNA interactions contributing to iron homeostasis. RNA 2016, 22 (8), 1271-82.
35. Mazzulli, J. R.: Zunke, F.: Isacson, O.; Studer, L.: Krainc, D., alpha-Synuclein-induced lysosomal dysfunction occurs through disruptions in protein trafficking in human midbrain synucleinopathy models. Proc. Natl. Acad. Sci. U.S.A. 2016, 113 (7), 1931-6.
36. Baksi, S.: Singh, N., alpha-Synuclein impairs ferritinophagy in the retinal pigment epithelium: Implications for retinal iron dyshomeostasis in Parkinson's disease. Sci. Rep. 2017, 7 (1), 12843.
37. Jean, J. M.; Hall, K. B., 2-Aminopurine fluorescence quenching and 5 lifetimes: role of base stacking. Proc. Natl. Acad. Sci. U.S.A. 2001, 98 (1), 37-41.
38. Kaul, M.; Barbieri, C. M.: Pilch, D. S., Fluorescence-based approach for detecting and characterizing antibiotic-induced conformational changes in ribosomal RNA: comparing aminoglycoside binding to prokaryotic and eukaryotic ribosomal RNA sequences. J. Am. Chem. Soc. 2004, 126 (11), 3447-53.
39. Shandrick, S.; Zhao. Q.; Han, Q.; Ayida, B. K.; Takahashi. M.; Winters, G. C.; Simonsen, K. B.; Vourloumis, D.; Hermann, T., Monitoring molecular recognition of the ribosomal decoding site. Angew. Chem. Int. Ed. Engl. 2004, 43 (24), 3177-82.
40. Liu, B.; Childs-Disney, J. L.; Znosko, B. M.; Wang, D.; Fallahi, M.; Gallo, S. M.: Disney, M. D., Analysis of secondary structural elements in human microRNA hairpin precursors. BMC Bioinformatics 2016, 17, 112.
41. Disney, M. D., Targeting RNA with small molecules to capture opportunities at the Intersection of chemistry, biology, and medicine. J. Am. Chem. Soc. 2019, 141 (17), 6776-6790.
42. Wager, T. T.; Hou, X.: Verhoest, P. R.; Villalobos, A., Central nervous system multiparameter optimization desirability: application in drug discovery. ACS Chem. Neurosci. 2016, 7 (6), 767-75.
43. Disney, M. D.; Dwyer, B. G.: Childs-Disney, J. L., Drugging the RNA World. Cold Spring Harb. Perspect. Biol. 2018, 10 (11).
44. Yang, W. Y.; Wilson, H. D.; Velagapudi, S. P.; Disney, M. D., Inhibition of non-ATG translational events in cells via covalent small molecules targeting RNA. J. Am. Chem. Soc. 2015, 137 (16), 5336-45.
45. Zarrinkar, P. P.; Wang, J.; Williamson. J. R., Slow folding kinetics of RNase P RNA. RNA 1996, 2 (6), 564-73.
46. Zarrinkar, P. P.: Williamson, J. R., Kinetic intermediates in RNA folding. Science 1994, 265 (5174), 918-24.
47. Chasse, H.: Boulben, S.; Costache, V.; Cormier. P.; Morales, J., Analysis of translation using polysome profiling. Nucleic Acids Res. 2017, 45 (3), e15.
48. Pena, C.; Hurt, E.; Panse. V. G., Eukaryotic ribosome assembly, transport and quality control. Nat. Struct. Mol. Biol. 2017, 24 (9), 689-699.
49. Eisenstein, R. S., Iron regulatory proteins and the molecular control of mammalian iron metabolism. Annu. Rev. Nutr. 2000, 20, 627-62.
50. Hentze, M. W.; Kuhn, L. C., Molecular control of vertebrate iron metabolism: mRNA-based regulatory circuits operated by iron, nitric oxide, and oxidative stress. Proc. Natl. Acad. Sci. U.S.A 1996, 93 (16), 8175-82.
51. Guo, B.; Phillips, J. D.; Yu, Y.; Leibold, E. A., iron regulates the intracellular degradation of iron regulatory protein 2 by the proteasome. J. Biol. Chem. 1995, 270 (37), 21645-51.
52. Devi, L.; Raghavendran, V.; Prabhu, B. M.; Avadhani, N. G.; Anandatheerthavarada, H. K., Mitochondrial import and accumulation of alpha-synuclein impair complex I in human dopaminergic neuronal cultures and Parkinson disease brain. J. Biol. Chem. 2008, 283 (14), 9089-100.
53. Liu, G.; Zhang, C.; Yin, J.; Li, X.; Cheng, F.; Li, Y.; Yang, H.; Ueda, K.; Chan, P.; Yu, S., alpha-Synuclein is differentially expressed in mitochondria from different rat brain regions and dose-dependently down-regulates complex I activity. Neurosci. Lett. 2009, 454 (3), 187-92.
54. Stojic, L.; Lun, A. T. L.; Mangei, J.; Mascalchi, P.; Quarantotti, V.; Barr, A. R.; Bakal, C.; Marioni, J. C.; Gergely, F.; Odom, D. T., Specificity of RNAi, LNA and CRISPRi as loss-of-function methods in transcriptional analysis. Nucleic Acids Res. 2018, 46 (12), 5950-5966.
55. Bandyopadhyay, S.; Cahill, C.; Balleidier, A.; Huang, C.; Lahiri, D. K.; Huang, X.; Rogers, J. T., Novel 5′ untranslated region directed blockers of iron-regulatory protein-1 dependent amyloid precursor protein translation: implications for down syndrome and Alzheimer's disease. PLoS One 2013, 8 (7), e65978.
56. Rumble, B.; Retallack, R.; Hilbich, C.; Simms, G.; Multhaup, G.; Martins, R.; Hockey, A.; Montgomery, P.; Beyreuther, K.; Masters, C. L., Amyloid A4 protein and its precursor in Down's syndrome and Alzheimer's disease. N. Engl. J. Med. 1989, 320 (22), 1446-52.
57. Su, Z.: Zhang, Y.; Gendron, T. F.: Bauer, P. O.: Chew, J.: Yang, W. Y.: Fostvedt, E.; Jansen-West, K.: Belzil, V. V.: Desaro. P.; Johnston, A., Overstreet, K.; Oh, S. Y.; Todd, P. K.; Berry, J. D.; Cudkowicz, M. E.; Boeve, B. F.: Dickson, D.; Floeter, M. K.: Traynor, B. J.: Morelli, C.; Ratti, A.; Silani, V.; Rademakers, R.; Brown, R. H.; Rothstein, J. D.; Boylan, K. B.; Petrucelli, L.; Disney, M. D., Discovery of a biomarker and lead small molecules to target r(GGGGCC)-associated defects in c9FTD/ALS. Neuron 2014, 83 (5), 1043-50.
58. Yang, W. Y.; He. F.; Strack, R. L.; Oh, S. Y.: Frazer, M.: Jaffrey, S. R.: Todd, P. K.; Disney, M. D., Small molecule recognition and tools to study modulation of r(CGG)^{exp} in fragile X-associated tremor ataxia syndrome. ACS Chem. Biol. 2016, 1 (9), 2456-65.
59. Vo, D. D.; Duca, M., Design of multimodal small molecules targeting miRNAs biogenesis: synthesis and in vitro evaluation. Methods Mol. Biol. 2017, 1517, 137-154.
60. Vo, D. D.; Becquart, C.; Tran, T. P. A.: Di Giorgio, A.; Darfeuille, F.: Staedel, C.: Duca, M., Building of neomycin-nucleobase-amino acid conjugates for the inhibition of oncogenic miRNAs biogenesis. Org. Biomol. Chem. 2018, 16 (34), 6262-6274.
61. Murata. A.; Otabe, T.; Zhang, J.; Nakatani, K., BzDANP, a small-molecule modulator of pre-miR-29a maturation by Dicer. ACS Chem. Biol. 2016, 11 (10), 2790-2796.
62. Murata, A.; Harada, Y.; Fukuzumi, T.: Nakatani, K., Fluorescent indicator displacement assay of ligands targeting 10 microRNA precursors. Boorg. Med. Chem. 2013, 21 (22), 7101-6.
63. Davidson, A.; Leeper, T. C.; Athanassiou, Z.; Patora-Komisarska, K.; Karn, J.; Robinson, J. A.: Varani, G., Simultaneous recognition of HIV-1 TAR RNA bulge and loop sequences by cyclic peptide mimics of Tat protein. Proc. Natl. Acad. Sci. U.S.A. 2009, 106 (29), 11931-6.
64. Hamy, F.; Felder, E. R.: Heizmann, G.; Lazdins, J.; Aboul-ela, F.; Varani, G.: Karn, J.: Klimkait, T., An inhibitor of the Tat/TAR RNA interaction that effectively suppresses HIV-1 replication. Proc. Natl. Acad. Sci. USA. 1997, 94 (8), 3548-53.
65. Tan, R.; Chen, L.; Buettner, J. A.; Hudson, D.; Frankel, A. D., RNA recognition by an isolated alpha helix. Cell 1993, 73 (5), 103140.
66. Andrews, R. J.; Baber, L.; Moss, W. N., RNAStructuromeDB: A genome-wide database for RNA structural inference. Sci. Rep. 2017, 7.
67. O'Leary, C. A.; Andrews, R. J.; Tompkins, V. S.; Chen, J. L.; Childs-Disney, J. L.; Disney, M. D.; Moss, W. N., RNA structural analysis of the MYC mRNA reveals conserved motifs that affect gene expression. PLoS One 2019, 14 (6), e0213758.
68. Sickmeier, M.; Hamilton, J. A.; LeGall, T.; Vacic, V.; Cortese, M. S.; Tantos, A.; Szabo, B.; Tompa, P.; Chen, J.; Uversky, V. N.; Obradovic, Z.; Dunker, A. K., DisProt: the database of disordered proteins. Nucleic Acids Res. 2007, 35 (Database issue), D786-93.
69. Andrews, R J.; Roche, J.; Moss, W. N., ScanFold: an approach for genome-wide discovery of local RNA structural elements-applications to Zika virus and HIV. Peer J 2018, 6, e6136.
70. Davis, M.; Sagan, S. M.; Pezacki, J. P.; Evans, D. J.; Simmonds, P., Bioinformatic and physical characterizations of genome-scale ordered RNA structure in mammalian RNA viruses. J. Virol. 2008, 82 (23), 11824-36.
71. Pawlica, P.; Moss, W. N.; Steitz, J. A., Host miRNA degradation by Herpesvirus saimiri small nuclear RNA requires an unstructured interacting region. RNA 2016, 22 (8), 1181-9.
72. Lee, M. M.; French, J. M.; Disney, M. D., Influencing uptake and localization of aminoglycoside-functionalized peptoids. Mol. Biosyst. 2011, 7 (8), 2441-51.
73. Childs-Disney, J. L.; Tsitovich, P. B.; Disney, M. D., Using modularly assembled ligands to bind RNA internal loops separated by different distances. Chembiochem 2011, 12(14), 2143-6.
74. Bernat, V.: Disney, M. D., RNA structures as mediators of neurological diseases and as drug targets. Neuron 2015, 87 (1), 28-46.
75. Lee, J.; Park, E. H.; Couture, G.; Harvey, I.; Garneau, P.; Pelletier, J., An upstream open reading frame impedes translation of the huntingtin gene. Nucleic Acids Res 2002, 30 (23), 5110-9.
76. Pelletier, J.: Sonenberg, N., Insertion mutagenesis to increase secondary structure within the 5′ noncoding region of a eukaryotic mRNA reduces translational efficiency. Cell 1985, 40 (3), 515-26.
77. Lee, B. R.; Kamitani, T., Improved immunodetection of endogenous alpha-synuclein. PLoS One 2011, 6 (8), e23939.
78. Weinreb, P. H.; Zhen, W.; Poon, A. W.; Conway, K. A.; Lansbury, P. T., Jr., NACP, a protein implicated in Alzheimer's disease and learning, is natively unfolded. Biochemistry 1996, 35 (43), 13709-15.
79. Yan, R.; Zhang, J.; Park, H. J.; Park, E. S.; Oh, S.; Zheng, H.; Junn, E.; Voronkov, M.; Stock, J. B.; Mouradian, M. M., Synergistic neuroprotection by coffee components eicosanoyl-5-hydroxytryptamide and caffeine in models of Parkinson's disease and DLB. Proc. Natl. Acad. Sci. U.S.A 2018,115 (51), E12053-E12062.

### MATERIALS AND METHODS REFERENCES

1. S. P. Velagapudi, S. J. Seedhouse, J. French, M. D. Disney, Defining the RNA internal loops preferred by benzimidazole derivatives via 2D combinatorial screening and computational analysis. J. Am. Chem. Soc. 133, 10111-10118 (2011).
2. K. K. Harris et al., Novel imidazoline antimicrobial scaffold that inhibits DNA replication with activity against mycobacteria and drug resistant Gram-positive cocci. ACS Chem. Biol. 9, 2572-2583 (2014).
3. B. Li et al., Synthesis and biological evaluation of botulinum neurotoxin a protease inhibitors. J. Med. Chem. 53, 2264-2276 (2010).
4. D. Maiti, B. P. Fors, J. L. Henderson, Y. Nakamura, S. L. Buchwald, Palladium-catalyzed coupling of functionalized primary and secondary amines with aryl and heteroaryl halides: two ligands suffice in most cases. Chem. Sci. 2, 57-68 (2011).
5. R. Lorenz et al., ViennaRNA Package 2.0. Algorithms Mol. Biol. 6, 26 (2011).
6. B. R. Lee, T. Kamitani, Improved immunodetection of endogenous alpha-synuclein. PLoS One 6, e23939 (2011).
7. P. H. Weinreb, W. Zhen, A. W. Poon, K. A. Conway, P. T. Lansbury, Jr., NACP, a protein implicated in Alzheimer's disease and learning, is natively unfolded. Biochemistry 35, 13709-13715 (1996).
8. R. Yan et al., Synergistic neuroprotection by coffee components eicosanoyl-5-hydroxytryptamide and caffeine in models of Parkinson's disease and DLB. Proc. Natl. Acad Sci. U.S.A. 115, E12053-E12062 (2018).
9. W. Y. Yang, H. D. Wilson, S. P. Velagapudi, M. D. Disney, Inhibition of non-ATG translational events in cells via covalent small molecules targeting RNA. J. Am. Chem. Soc. 137, 5336-5345 (2015).
10. W. Y. Yang et al, Small molecule recognition and tools to study modulation of r(CGG)^{exp} in fragile X-associated tremor ataxia syndrome. ACS Chem. Biol. 11, 2456-2465 (2016).
11. J. Cox, M. Mann, MaxQuant enables high peptide identification rates, individualized p.p.b.-range mass accuracies and proteome-wide protein quantification. Nat. Biotechnol. 26, 1367-1372 (2008).
12. N. L. Bray, H. Pimentel, P. Melsted, L. Pachter, Near-optimal probabilistic RNA-seq quantification. Nat. Biotechnol. 34, 525-527 (2016).
13. H. Pimentel, N. L. Bray, S. Puente, P. Melsted, L. Pachter, Differential analysis of RNA-seq incorporating quantification uncertainty. Nat. Methods 14, 687-690 (2017).
14. D. Piovesan et al., DisProt 7.0: a major update of the database of disordered proteins. Nucleic Acids Res. 45, D219-D227 (2017).
15. R J. Andrews, J. Roche, W. N. Moss, ScanFold: an approach forgenome-wide discovery of local RNA structural elements-applications to Zika virus and HIV. Peer J 6, e6136 (2018).
16. J. T. Robinson et al., Integrative genomics viewer. Nat. Biotechnol. 29, 24-26(2011).
17. J. T. Rogers et al., The alpha-synuclein 5′untranslated region targeted translation blockers: anti-alpha synuclein efficacy of cardiac glycosides and Posiphen. J. Neural Transm. 118, 493-507 (2011).

### SUMMARY STATEMENTS

The inventions, examples, biological assays and results described and claimed herein have may attributes and embodiments include, but not limited to, those set forth or described or referenced in this application.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

1. A method for complexing an SNCA mRNA comprising contacting the SNCA mRNA with a synucleozid compound comprising Formula I 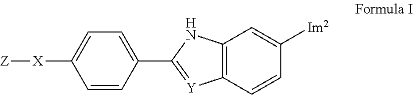
wherein:
Y is nitrogen or CR¹
X is oxygen or NR²;
Z is hydrogen, methyl or 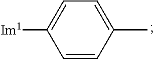
Im¹ is guanidyl 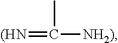
imidazolyl, dihydroimidazolyl, imidazolinyl, pyrrolyl, pyrrolidinyl, or cyano;
Im² is guanidyl, imidazolyl, dihydroimidazolyl, imidazolinyl, pyrrolyl, pyrrolidinyl, or cyano;
Im¹ and Im² are the same or different;
R¹ and R² are each independently hydrogen or methyl;
and a pharmaceutically acceptable salt thereof.

2. A method according to claim 1 wherein the synucleozid compound of Formula I has Z as 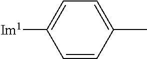
so that Formula I comprises Formula II 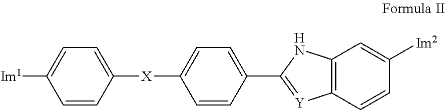
wherein R² is hydrogen and Im¹ and Im² are the same or different; and a pharmaceutically acceptable salt thereof.

3. A method of claim 1 wherein the SNCA mRNA is in a mixture with other mRNA molecules.

4. (canceled)

5. A method of claim 1 wherein the SNCA mRNA is present in a cell.

6. A method of claim 5 wherein the cell is in a cell culture

7. A method of claim 5 wherein the cell is in living tissue.

8. A method of claim 2 wherein the synucleozid compound is Formula II and Im¹ and Im² are each imidazolyl, dihydroimidazolyl, imidazolinyl, or guanidyl.

9.-11. (canceled)

12. A method of claim 2 wherein the synucleozid compound is Formula II and Y is N.

13. A method of claim 2 wherein the synucleozid compound is Formula II and Y is CH.

14. A method of claim 2 wherein the synucleozid compound is Formula II and X is O.

15. A method of claim 2 wherein the synucleozid compound is Formula II and X is NH.

16. A pharmaceutical composition comprising a pharmaceutical carrier and a synucleozid compound of Formula I of claim 1.

17. (canceled)

18. A method of claim 1 wherein the contacting step is an in vitro step with isolated mRNA.

19. A method for treatment of a synucleinopathy disease comprising administration to a patient having the disease, an effective amount of a synucleozid compound of Formula I of claim 1.

20. (canceled)

21. A method for treatment according to claim 19 wherein the synucleinopathy disease is Parkinson's disease, Dementia with Lewy Bodies or Multiple System Atrophy.

22. A method according to claim 21 wherein the disease is Parkinson's disease.

23. A method for reducing or inhibiting production of α-synuclein protein by a cell carrying the SNCA gene comprising applying to the cell a synucleozid compound of Formula I of claim 1.

24. A method according to claim 23 wherein the cell carrying the SNCA gene is present in living tissue.

25. A method according to claim 24 wherein the living tissue is tissue of a mammalian animal.

26. (canceled)

27. A method for reducing or inhibiting translation of synuclein messenger RNA in an extracellular medium also containing ribosomes comprising contacting the medium with a synucleozid compound of Formula I of claim 1.

28. A method for reducing or inhibiting translation of synuclein messenger RNA in a cell carrying the SNCA gene comprising contacting the cell with a synucleozid compound of Formula I of claim 1.

29. A method of claim 28 wherein the cell carrying the SNCA gene is in a cellular culture.

30. A method of claim 28 wherein the cell carrying the SNCA gene is in living tissue.

31. A method of claim 30 wherein the living tissue is tissue of a mammalian animal.

32. (canceled)

33. A pharmaceutical composition of claim 16, wherein the synucleozid compound of Formula I is a synucleozid compound of Formula II 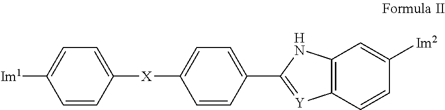
wherein
Y is nitrogen or CR¹
X is oxygen or NR²;
Z is hydrogen, methyl or 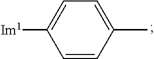
Im¹ is guanidyl 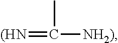
imidazolyl, dihydroimidazolyl, imidazolinyl, pyrrolyl, pyrrolidinyl, or cyano;
Im² is guanidyl, imidazolyl, dihydroimidazolyl, imidazolinyl, pyrrolyl, pyrrolidinyl, or cyano;
Im¹ and Im² are the same or different;
R¹ and R² are each independently hydrogen or methyl;
and a pharmaceutically acceptable salt thereof.

34. A composition according to claim 33 wherein Im¹ and Im² are the same.

35. A method according to claim 2 wherein Im¹ and Im² are the same.
